(19) United States
(12) Patent Application Publication
(10) Pub. No.: US 2003/0064413 A1
(43) Pub. Date: Apr. 3, 2003

(54) Compositions and methods useful in avidity therapy

(76) Inventor: Pere Santamaria, Calgary (CA)

Correspondence Address: BURNS DOANE SWECKER & MATHIS L L P, POST OFFICE BOX 1404, ALEXANDRIA, VA, 22313-1404, US

(21) Appl. No.: 10/005,917

(22) Filed: Dec. 7, 2001

Related U.S. Application Data
(60) Provisional application No. 60/254,304, filed on Dec. 8, 2000.

(51) Int. Cl. G01N 33/567; A61K 39/395; C12N 5/08

(52) U.S. Cl. 435/7.21; 435/372; 424/140.1

(57) ABSTRACT

This invention relates to compositions and methods useful for avidity therapy. Provided in particular are ligands which modulate the avidity maturation of T cell populations and are, thus, useful in the treatment of cancer and autoimmune diseases.

### RELATED APPLICATIONS

This application claims the benefit of U.S. Provisional Applications Serial No. 60/254,304, filed Dec. 8, 2000, the entire disclosure of which is hereby incorporated by reference.

### FIELD OF THE INVENTION

This invention relates to compositions and methods useful for avidity therapy. Provided in particular are ligands which modulate the avidity maturation of T-cell populations and are, thus, useful in the treatment of cancer and autoimmune diseases.

### REFERENCES

The following references are referred to in the specification by numbers in parentheses:

1. Tisch, R., and H. McDevitt. 1996. Insulin-dependent diabetes mellitus. Cell 85:291.

2. Delovitch, T., and B. Singh. 1997. The nonobese diabetic mouse as a model of autoimmune diabetes: immune disregulation gets the NOD. Immunity 7:727.

3. Hutchings, P., H. Rosen, L. O'Reilly, E. Simpson, S. Gordon, and A. Cooke. 1990. Transfer of diabetes in mice prevented by blockade of adhesion-promoting receptor on macrophages. Nature 348:639.

4. Lo, D., C. Reilly, B. Scott, R. Liblau, H. McDevitt, and L. Burkly. 1993. Antigen-presenting cells in adoptively transferred and spontaneous autoimmune diabetes. Eur. J. Immunol. 23:1693.

5. Jun, H. -S., P. Santamaria, H. -W. Lim, M. Zhang, and J. -W. Yoon. 1999. Absolute requirement of macrophages for the development and activation of B-Cell cytotoxic CD8⁺ T-Cells in T-Cell receptor transgenic NOD mice. Diabetes 48:34.

6. Jun, H., C. Yoon, L. Zbytnuik, N. v. Rooijen, and J. Yoon. 1999. The role of macrophages in T cell-mediated autoimmune diabetes in nonobese diabetic mice. J. Exp. Med. 189:347.

7. Christianson, S., L. Shultz, and E. Leiter. 1993. Adoptive transfer of diabetes into immunodeficient NOD-scid/scid mice. Relative contributions of CD4⁺ and CD8⁺ T-cells from diabetic versus prediabetic NOD.NON-thy-1a donors. Diabetes 42:44.

8. Bendelac, A., C. Carnaud, C. Boitard, and J. F. Bach. 1987. Syngeneic transfer of autoimmune diabetes from diabetic NOD mice to healthy neonates. J. Exp. Med. 166:823.

9. Miller, B. J., M. C. Appel, J. J. O'Neil, and L. S. Wicker. 1988. Both the Lyt-2⁺ and L3T4⁺ T cell subsets are required for the transfer of diabetes in non-obese diabetic mice. J. Immunol. 140:52.

10. Yagi, H., M. Matsumoto, K. Kunimoto, J. Kawaguchi, S. Makino, and M. Harada. 1992. Analysis of the roles of CD4⁺ and CD8⁺ T cells in autoimmune diabetes of NOD mice using transfer to NOD athymic nude mice. Eur. J. Immunol. 22:2387.

11. Thivolet, C., A. Bendelac, P. Bedossa, J. F. Bach, and C. Carnaud. 1991. CD8⁺ T cell homing to the pancreas in the nonobese diabetic mouse is CD4⁺ T cell-dependent. J. Immunol. 146:85.

12. Haskins, K., and M. McDuffie. 1990. Acceleration of diabetes in young NOD mice with a CD4⁺ islet-specific T cell clone. Science 249:1433.

13. Nakano, N., H. Kikutani, H. Nishimoto, and T. Kishimoto. 1991. T cell receptor V gene usage of islet beta cell-reactive T cells is not restricted in non-obese diabetic mice. J. Exp. Med. 173:1091.

14. Bradley, B., K. Haskins, F. L. Rosa, and K. Lafferty. 1992. CD8 T cells are not required for islet destruction induced by a CD4⁺ islet-specific T-cell clone. Diabetes 41:1603.

15. Daniel, D., R. Gill, N. Schloot, and D. Wegmann. 1995. Epitope specificity, cytokine production profile and diabetogenic activity of insulin-specific T cell clones isolated from NOD mice. Eur. J. Immunol. 25:1056.

16. Katz, J., C. Benoist, and D. Mathis. 1995. T helper cell subsets in insulin-dependent diabetes. Science 268:1185.

17. Peterson, J., and K. Haskins. 1996. Transfer of diabetes in the NOD-scid mouse by CD4 T cell clones. Differential requirement for CD8 T cells. Diabetes 45:328.

18. McInerney, M., S. Rath, and C. Janeway. 1991. Exclusive expression of MHC class II proteins on CD45⁺ cells in pancreatic islets of NOD mice. Diabetes 40:648.

19. Serreze, D., and E. Leiter. 1994. Genetic and pathogenic basis of autoimmune diabetes in NOD mice. Curr. Opin. Immunol. 6:900.

20. Wong, F., I. Visintin, L. Wen, R. Flavell, and J. CA Janeway. 1996. CD8 T cell clones from young NOD islets can transfer rapid onset of diabetes in NOD mice in the absence of CD4 T cells. J. Exp. Med. 183:67.

21. Katz, J., C. Benoist, and D. Mathis. 1993. Major histocompatibility complex class I molecules are required for the generation of insulitis in non-obese diabetic mice. Eur. J. Immunol. 23:3358.

22. Wicker, L., E. Leiter, J. Todd, R. Renjilian, E. Peterson, P. Fischer, P. Podolin, M. Zijlstra, R. Jaenisch, and L. Peterson. 1994. b2-microglobulin-deficient NOD mice do not develop insulitis or diabetes. Diabetes 43:500.

23. Serreze, D., E. Leiter, G. Christianson, D. Greiner, and D. Roopenian. 1994. Major histocompatibility complex class I-deficient NOD.blmₙᵤₗₗ mice are diabetes and insulitis resistant. Diabetes 43:505.

24. Wang, B., A. Gonzalez, C. Benoist, and D. Mathis. 1996. The role of CD8⁺ T-cells in initiation of insulin-dependent diabetes mellitus. Eur. J. Immunol. 26:1762.

25. Nagata, M., P. Santamaria, T. Kawamura, T. Utsugi, and J. -W. Yoon. 1994. Evidence for the role of CD8⁺ cytotoxic T cells in the destruction of pancreatic beta cells in NOD mice. Submitted.

26. Hayakawa, M., K. Yokono, M. Nagata, N. Hatamori, W. Ogawa, A. Miki, H. Mizoguti, and S. Baba. 1991. Morphological analysis of selective destruction of pancreatic beta cells by cytotoxic T lymphocytes in NOD mice. Diabetes 40:1210.

27. Nagata, M., and J. -W. Yoon. 1992. Studies on autoimmunity for T cell-mediated beta cell destruction: distinct difference in the destruction of beta cells between CD4⁺ and CD8⁺ T cell clones derived from lymphocytes infiltrating the islets of NOD mice. Diabetes 41:998.

28. Shimizu, J., O. Kanagawa, and E. Unanue. 1993. Presentation of beta cell antigens to CD4⁺ and CD8⁺ T cells of non-obese diabetic mice. J. Immunol. 151:1723.

29. Santamaria, P., T. Utsugi, B. Park, N. Averill, S. Kawazu, and J. Yoon. 1995. Beta cell cytotoxic CD8⁺ T cells from non-obese diabetic mice use highly homologous T cell receptor alpha chain CDR3 sequences. J. Immunol. 154:2494.

30. Verdaguer, J., J. -W. Yoon, B. Anderson, N. Averill, T. Utsugi, B. -J. Park, and P. Santamaria. 1996. Acceleration of spontaneous diabetes in TCRb-transgenic nonobese diabetic mice by beta cell-cytotoxic CD8⁺ T cells expressing identical endogenous TCRa chains. J. Immunol. 157:4726.

31. Kay, T., J. Parker, L. Stephens, H. Thomas, and J. Allison. 1996. RIP-b2-microglobulin transgene expression restores insulitis, but not diabetes, in b2-microglobulinₙᵤₗₗ nonobese diabetic mice. J. Immunol. 157:3688.

32. Serreze, D., H. Chapman, D. Varnum, I. Gerling, E. Leiter, and L. Shultz. 1997. Initiation of autoimmune diabetes in NOD/Lt mice is MHC class I-dependent. J. Immunol. 157.3978.

33. Vyse, T., and J. Todd. 1996. Genetic analysis of autoimmune disease. Cell 85:311.

34. Verdaguer, J., D. Schmidt, A. Amrani, B. Anderson, N. Averill, and P. Santamaria. 1997. Spontaneous autoimmune diabetes in monoclonal T cell nonobese diabetic mice. J. Exp. Med. 186:1663.

35. Schmidt, D., J. Verdaguer, N. Averill, and P. Santamaria. 1997. A mechanism for the major histocompatibility complex-linked resistance to autoimmunity. J. Exp. Med. 186:1059.

36. DiLorenzo, T., T. Graser, T. Ono, G. Christianson, H. Chapman, D. Roopenian, S. Nathenson, and D. Serreze. 1998. MHC class I-restricted T-cells are required for all but end stages of diabetes development and utilize a prevalent T cell receptor a chain gene rearrangement. Proc. Natl. Acad. Sci. USA 95:12538.

37. Anderson, B., B. -J. Park, J. Verdaguer, A. Amrani, and P. Santamaria. 1999. Dominant CD8⁺ T-cell response against a single peptide/MHC complex in autoimmune diabetes. Proc. Natl. Acad. Sci. USA 96:9311.

38. Wong, S., and e. al. 1999. Identification of an MHC class I-restricted autoantigen in type 1 diabetes by screening an organ-specific cDNA library. Nature Med. 5:1026.

39. Amrani, A., J. Verdaguer, P. Serra, S. Tafuro, R. Tan, and P. Santamaria. 2000. Progression of autoimmune diabetes driven by avidity maturation of a T-cell population. Nature. 406:739, 2000.

40. Utsugi, T., J. -W. Yoon, B. -J. Park, M. Imamura, N. Averill, S. Kawazu, and P. Santamaria. 1996. MHC class I-restricted infiltration and destruction of pancreatic islets by NOD mouse-derived beta cell-cytotoxic CD8⁺ T-cell clones in vivo. Diabetes 45:1121.

41. Amrani, A., J. Verdaguer, B. Anderson, T. Utsugi, S. Bou, and P. Santamaria. 1999. Perforin-independent beta cell destruction by diabetogenic CD8⁺ T lymphocytes in transgenic nonobese diabetic mice. J. Clin. Invest. 103:1201.

42. Kagi, D., B. Odermatt, P. Seiler, R. Zinkernagel, T. Mak, and H. Hengartner. 1997. Reduced incidence and delayed onset of diabetes in perforin-deficient nonobese diabetic mice. J. Exp. Med. 186:989.

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

Insulin-dependent diabetes mellitus (IDDM) is the result of a T-cell-mediated autoimmune process directed against the pancreatic beta cells (1, 2). IDDM studies are usually performed using NOD mice, which spontaneously develop a form of diabetes that resembles human IDDM (1, 2). IDDM in NOD mice also requires the presence of B-cells and macrophages (1-6).

The role of CD8⁺ T-cells in the initiation of IDDM has been studied by way of T-cell transfer studies using splenocytes from prediabetic NOD mice. These studies have shown that IDDM requires both CD4⁺ and CD8⁺ T-cells (7-17). Since beta cells do not express MHC class II molecules (18), it has been proposed that naive autoreactive CD4⁺ T-cells differentiate into effector cells by engaging beta cell antigens (shed by a prior insult) on local antigen-presenting cells (APCs) (19, 20). Studies of β2-microglobulin-deficient (β2m−) NOD mice and anti-CD8 mAb-treated NOD mice (21-24) have suggested that the initial insult that triggers the shedding of beta cell antigens is mediated by CD8⁺ cytotoxic T-cells (CTL), which are consistently present in NOD islets (20, 25-30). This view is compatible with two other findings: that restoring MHC class I expression on beta cells of β2m− NOD mice restores insulitis susceptibility (31), and that splenocytes from NOD mice cannot transfer insulitis into β2m− NOD.scid mice (32). This hypothesis, however, is in conflict with observations suggesting that IDDM is “initiated”by CD4⁺ T-cells. For example, splenic CD4⁺ T-cells from NOD mice can transfer insulitis into NOD.scid mice, but splenic CD8⁺ T-cells cannot (7, 11). Furthermore, susceptibility to insulitis and IDDM is profoundly affected by polymorphisms of MHC class II genes (1, 33), which control the development and function of CD4⁺, but not CD8⁺, T-cells.

Results of previous studies in our laboratory support the view that efficient accumulation of CD8⁺ pre-CTL into islets requires CD4⁺ T-cell help. This was determined by comparing the ability of NOD islet-derived, I-_{Ag7}- or _{Kd}-restricted, beta cell-specific T-cell receptors (TCRs) (named 4.1 and 8.3, respectively) to trigger IDDM in TCR-transgenic/recombination activating gene 2-deficient (RAG-2^{-/-}) NOD mice, which cannot rearrange endogenous TCR genes. These studies showed that, unlike naive diabetogenic CD4⁺ T-cells, naive diabetogenic CD8⁺ T-cells are largely dependent on endogenous (CD4⁺ ) T-cell help to accumulate efficiently in islets (34). Since 4.1-CD4⁺ thymocytes undergo deletion in IDDM-resistant NOD.H-2_{g7/x} mice by engaging anti-insulitogenic MHC class II molecules on thymic APCs (35), we have proposed that initiation of IDDM requires both an “initial” beta cell insult by 4.1-like CD4⁺ T-cells and the immediate recruitment of certain CD8⁺ T-cells. As pointed out in reference 34, the fact that IDDM in RAG-2^{-/-} 4.1-NOD mice bypasses the need for CD8⁺ T-cells does not contradict this view; the high frequency of autoreactive CD4⁺ T-cells in 4.1-NOD mice likely overwhelms the mechanisms that, in non-transgenic mice, would prevent these cells from reaching an insulitogenic mass upon activation.

The mechanisms which drive the recruitment of CD8⁺ T-cells to islets have also been explored. The antigenic specificity(ies) of the CD8⁺ T-cells involved in the initiation and/or progression of IDDM is (are) unknown. Several lines of evidence suggest that the antigenic repertoire of these T-cells is very restricted. Most of the CD8⁺ T-cells that can be isolated from islets of acutely diabetic NOD mice are cytotoxic to beta cells in the context of the MHC class I molecule K_{d}, and use TCRα chains with homologous CDR3 sequences (29). Furthermore, the majority of the islet-associated CD8⁺ T-cells of transgenic NOD mice expressing the TCRβ chain of the CD8⁺ clone NY8.3 (which uses a representative CDR3α sequence) express an endogenously-derived TCRα chain that is identical to the one employed by the clonotype donating the TCRβ transgene (30). Strikingly, DiLorenzo et al. (36) recently showed that a significant % of the CD8⁺ T-cells that can be propagated from the earliest insulitic lesions of NOD mice (at 4-5 wk) use TCRα chains that are very similar, or even identical, to those used by the CD8⁺ T-cells that we had isolated from diabetic NOD mice and 8.3-TCRβ-transgenic NOD mice (Vα17 and Jα42 elements joined by the N-region sequence MRD/E). This TCRαβ heterodimer is highly pathogenic, since 8.3-TCRαβ-transgenic NOD mice develop diabetes shortly after the onset of insulitis (34).

By screening combinatorial peptide libraries, we identified two peptide ligands for CTL expressing the prevalent Vα17-MRD-Jα42 TCRα chain: NRP (KYNKANWFL) and NRP-A7 (KYNKANAFL), an alanine mutant analog of NRP with superior agonistic properties (37). These two peptides elicit the proliferation, cytokine secretion, differentiation and cytotoxicity of naive CD8⁺ T-cells from 8.3-TCRαβ-transgenic NOD mice, and are recognized by a large fraction of the islet-associated CD8⁺ T-cell lines (87%) and clones (45%) recruited to islets in NOD mice. This observation has thus provided additional support to our view that activation of the CD8⁺ T-cells that contribute to the progression of spontaneous IDDM is triggered by recognition of a few peptide/MHC class I complexes on beta cells or islet-associated APCs (37). It is important to point out, however, that this does not imply that the CD8⁺ T-cell response in autoimmune diabetes is exclusively directed against one peptide. Wong et al., for example, have reported that insulitic CD8⁺ T-cells in young NOD mice recognize an insulin-derived peptide (38). In our hands, however, these cells represent a very small fraction of all islet-derived CD8⁺ T-cells, particularly in mice older than 6 weeks [see below and reference 39].

There is now ample evidence indicating that CD8⁺ CTL also function as major effectors of beta cell lysis in IDDM, along with CD4⁺ T-cells. CD8⁺ CTL are consistently present in islets of NOD mice (20, 25-28), can transfer IDDM into NOD.scid mice (20, 25), and can kill beta cells of IDDM-resistant mice in vivo (40). Our study of NOD mice expressing the 8.3-TCRβ transgene provided in vivo evidence for a major contribution of CD8⁺ CTL to beta cell loss in spontaneous IDDM: these mice have a relatively minor (but selective) increase in the frequency of beta cell-reactive CD8⁺ CTL precursors and develop accelerated IDDM, owing to an accelerated recruitment of CD8⁺ (but not CD4⁺ ) T-cells to islets (30). Although 8.3-CD8⁺ CTL kill beta cells via Fas exclusively (41), studies of perforin-deficient NOD mice have demonstrated that CD8⁺ CTL clonotypes that are recruited to islets later on in the disease process need to express perforin to kill beta cells (42).

To date, IDDM is primarily treated by monitoring blood glucose level and administrating insulin when glucose level is low, in order to compensate for beta cells that are damaged by T cells as described above. This treatment is tedious and does not solve the fundamental problem of beta cell loss. Therefore, the need remains for a more fundamental approach to treating IDDM.

### SUMMARY OF THE INVENTION

The present invention relates to a method of selectively reducing or expanding T cells according to the antigenic specificity of the T cells. Therefore, the present invention can be used to reduce or eliminate T cells that recognize auto-antigens, such as beta cell specific T cells. As such, the present invention can be used to prevent, treat or ameliorate autoimmune diseases such as IDDM. Furthermore, the present invention can be used to expand desirable T cells, such as T cells that recognize tumor antigens, to prevent, treat and/or ameliorate diseases battled by these T cells.

Accordingly, one aspect of the present invention provides a method for selectively expanding or deleting at least one T cell from a T cell population, comprising:

(a) providing a ligand that binds to at least one T cell in said T cell population with a desired avidity; and

(b) contacting said T cell population with an effective amount of said ligand under conditions wherein the T cells that bind to said ligand with an avidity higher than the desired avidity are deleted, the T cells that bind to said ligand with an avidity lower than the desired avidity are expanded, and the T cells that do not bind said ligand are unaffected.

The ligand can be prepared by the steps comprising:

(i) providing a test ligand which is recognized by said at least one T cell in said T cell population;

(ii) preparing a series of ligand mimics based on said test ligand;

(iii) determining the binding avidity of said test ligand and said series of ligand mimics to said at least one T cell; and

(iv) selecting the ligand mimics in said series of ligand mimics that bind to said at least one T cell with the desired avidity.

The binding avidity is preferably tested by using a tetramer of the complex between the ligand mimic and the MHC molecule of interest.

In one embodiment of this method, the T cells deleted in step (b) are auto-reactive T cells, particularly those mediating IDDM. The preferred ligand in this embodiment is selected from the group consisting of NRP-A4, NRP-I4, NRP, NRP-A7 and NRP-V7.

In another embodiment of this method, the T cells expanded in step (b) recognize pathogenic or tumor antigens. Expansion of these T cells thus is useful in the treatment of pathogenic diseases and tumors.

The ligand can be any molecule capable of binding specifically to T cell receptors. Preferably, the ligand is a peptide.

Another aspect of the present invention provides a method of screening for a ligand that is capable of deleting or expanding at least one T cell from a T cell population, comprising:

(i) providing a test ligand which is recognized by said at least one T cell in said T cell population;

(ii) preparing a series of ligand mimics based on said test ligand;

(iii) determining the binding avidity of said test ligand and said series of ligand mimics to said at least one T cell; and

(iv) selecting a ligand mimics in said series of ligand mimics that bind to said at least one T cell with a desired avidity.

The binding avidity is preferably tested by using a tetramer of the complex between the ligand mimic and the MHC molecule of interest.

Another aspect of the present invention provides a method of preventing, ameliorating or treating an autoimmune disease which is caused by at least one auto-reactive T cell in a mammal, comprising:

(a) providing a ligand which binds to said at least one auto-reactive T cell with a desired avidity; and

(b) administering to said mammal an effective amount of said ligand under conditions wherein the T cells which bind to said ligand with an avidity higher than the desired avidity are deleted, the T cells which bind to said ligand with an avidity lower than the desired avidity are expanded, and the T cells which do not bind said ligand are unaffected.

Another aspect of the present invention provides a method of diagnosing an autoimmune disease mediated by a T cell, comprising combining a biological sample with a ligand known to bind the T cells mediating the autoimmune disease, wherein the presence of T cells in the biological sample capable of binding the ligand indicates a positive diagnosis for the disease. The binding activity is preferably tested by using a tetramer of the complex between the ligand and the MHC molecule of interest.

Yet another aspect of this invention provides a composition useful for selectively expanding a T cell clone or deleting a T cell clone, comprising a ligand which binds to at least one T cell in a T cell population with a desired avidity; wherein contacting said T cell population with an effective amount of said ligand results in deletion of the T cells which bind to said ligand with an avidity higher than the desired avidity, and expansion of the T cells which bind to said ligand with an avidity lower than the desired avidity, while T cells which do not bind said ligand are unaffected. The ligand is preferably a peptide, more preferably a peptide selected from the group consisting of NRP-A4, NRP-I4, NRP, NRP-A7 and NRP-V7. Also provided are pharmaceutical compositions comprising the compositions described above.

Another aspect of the present invention provides multimers of a ligand-MHC complex. These multimers are useful, for example, in binding assays for the identification of ligands with desired avidities. The multimer is preferably a dimer or tetramer, and more preferably a tetramer.

### BRIEF DESCRIPTION OF THE DRAWINGS

FIG. 1 illustrates that, in 8.3-NOD, but not 8.3-TCRβ-NOD, transgenic NOD mice expressing the TCRα and β chains of NY8.3 (8.3-NOD mice), thymocyte development was skewed towards the CD8⁺ subset, as expected (34).

FIG. 2 demonstrates that the splenic CD8⁺ T cells of 8.3-NOD mice proliferated in response to NOD islet cells in vitro (FIG. 2A) owing to a 400-fold increase in the peripheral frequency of beta cell-reactive CD8⁺ T cells (FIG. 2B).

FIG. 3 illustrates the development of IDDM, showing that these mice developed IDDM much earlier than NOD or 8.3-TCRβ-transgenic NOD mice (FIG. 3A), coinciding with an accelerated recruitment of CD8⁺ CTL (FIGS. 3B-D).

FIG. 4 illustrates results of experiments using monoclonal T cell NOD mice. 8.3-TCR transgenes were introduced into RAG-2^{-/-} NOD mice (FIG. 4A). RAG-2^{-/-} 8.3-NOD mice also developed IDDM, but they did so less frequently, and later than RAG-2⁺ 8.3-NOD mice (FIG. 4B). Nearly all the islet-infiltrating T cells in the diabetic mice were either CD4⁺ (in RAG-2^{-/-} 4.1-NOD mice) or CD8⁺ (in RAG-2^{-/-} 8.3-NOD mice) (FIG. 4C).

FIG. 5 shows the effect of endogenous T cell help on 8.3-CD8⁺ T cells.

FIG. 6 illustrates that NRP had the same agonistic properties as the natural beta cell auto-antigen on naive 8.3-CD8⁺ T cells.

FIG. 7 presents the NRP-reactivity of islet-associated CD8⁺ T cells from NOD mice (37). As shown in FIG. 7A, most CD8⁺ T cells propagated from islets of acutely diabetic NOD mice (7 of 8—87.5%—, FIG. 7C) killed NRP-[but not negative control peptide (TUM)]-pulsed RMA-SK_{d} cells in vitro. Of 31 CD8⁺ CTL clones generated from islets of 9 NOD mice which were tested, 14 (45%) were cytotoxic against NRP-, but not TUM-pulsed, RMA-SK_{d} targets (FIGS. 7B and C).

FIG. 8 illustrates that tetramer staining indicated that NRP/NRP-A7-reactive CD8⁺ T cells constitute a significant fraction of the in vivo-activated CD8⁺ T cells contained within islets (FIGS. 8A, B) and that the size of the NRP-A7-reactive CD8⁺ T cell population increased with age and was antigen-specific. Most, if not all the NRP-reactive lines are also NRP-A7-reactive, but not vice versa (FIG. 8C).

FIG. 9 demonstrates that the above result (FIG. 8) was not an artifact of in vitro culture since pancreatic lymph node cells from 20 wk-old, but not 3-5 wk-old NOD mice bind the NRP-A7 tetramer more efficiently than the NRP tetramer (FIG. 9).

FIG. 10 illustrates that at least one of the Vα17-MRD-Jα42-expressing T cell clones isolated by DiLorenzo et al. (36) recognizes NRP-A7 but not NRP. FIG. 11 shows that NRP/NRP-A7-reactive clonotypes (i.e. 8.3-CTL) bind the NRP-A7 tetramer with longer half-life and higher avidity (lower K_{D}) than the NRP tetramer (FIGS. 11A and 11B), indicating that the outgrowing clonotypes might be those with the highest avidity for NRP-A7/H-2K_{d} binding. As shown in FIG. 11C, CD8⁺ T cells isolated from 20 wk-old NOD mice bound the NRP-A7 tetramer with higher avidity and longer half-life than CD8⁺ T cells isolated from 9 wk-old mice. The islet-associated NRP-A7-reactive CD8⁺ T cell population therefore increases its avidity for peptide/MHC as the mice approach the age at which the incidence of IDDM follows an exponential distribution (FIG. 11D).

FIG. 12 illustrates TUM-treated NOD mice developed IDDM with an incidence similar to untreated NOD mice. NOD mice treated with NRP developed IDDM at a slightly lower rate when compared to TUM-treated mice while mice treated with NRP-A7 were highly protected (FIG. 12A). Pathological studies revealed that TUM-, NRP- and NRP-A7-treated mice displayed similar insulitis scores (FIG. 12B). The islet-derived CD8⁺ T cells of NRP-A7-and NRP-treated mice contained greater numbers of NRP-A7 tetramer-reactive cells than control peptide-treated mice (FIGS. 12C and 12D). However, the CD8⁺ T cells derived from NRP-A7-treated mice were less cytotoxic to NRP-A7-pulsed, RMA-SK_{d} targets than those derived from NRP- or control peptide-treated mice (FIG. 12E). As shown in FIG. 12F, CD8⁺ T cells derived from NRP-A7-treated mice bound the NRP-A7 tetramer with a lower avidity and shorter half-life than CD8⁺ T cells derived from NRP- or control peptide-treated mice.

FIG. 13 summarizes the results of experiments conducted by generating numerous single amino acid mutants of NRP (NRP mimics) and testing the ability of each mutant to elicit proliferation, cytokine secretion and cytotoxicity of naïve or activated 8.3-CD8⁺ T cells.

FIG. 14 describes the activity of two NRP antagonists: NRP-A4 and NRP-A8

FIG. 15 shows a comparison of the agonistic properties of various peptides on naïve 8.3-CD8⁺ T cells.

FIG. 16 illustrates that the NRP-A4 tetramer (antagonist) interacted with the 8.3-TCR TCR on 8.3-CD8⁺ T cells with an avidity that was too low to stably stain 8.3-CD8⁺ T cells.

FIG. 17 shows that the NRP-V7 tetramer reacts with as many, and sometimes more islet-derived CD8⁺ T cells than the NRP-A7 tetramer, much like what we saw with the NRP and NRP-A7 tetramers (more cells bound the NRP-A7 than the NRP tetramers).

FIG. 18 illustrates that NRP-I4 not only did not protect the mice from diabetes, but actually caused disease acceleration.

FIG. 19 shows that the pancreatic islets of the NRP-I4 treated mice contained more -A7-reactive CD8⁺ T cells than untreated NOD mice, and their cells were capable of binding the NRP-A7, but not the NRP tetramer.

FIG. 20 provides kinetic analyses which showed that these cells bound NRP-A7 tetramer with high avidity.

FIGS. 21 and 22 illustrate that the islet-associated CD8⁺ T cells of NRP-V7-treated mice had virtually no NRP-reactive CD8⁺ T cells, as determined by tetramer staining.

FIG. 23 provides a summary of the mechanism of modulation and criteria for peptide selection.

FIG. 24 illustrates a model for the proposed relationship between avidity/affinity of peptides for TCRs and their therapeutic potential.

FIG. 25 shows the effects of low affinity NRP mimics (NRP-A4 and NRP-I4) on the onset and percentage of Type 1 Diabetes (T1D) incidence. “Low” and “High” indicate the dosages of the peptide.

FIG. 26 shows the effects of high affinity NRP mimics (NRP-V7 and NRP-A7) on the onset and percentage of Type 1 Diabetes (T1D) incidence. “Low” and “High” indicate the dosages of the peptide.

FIG. 27 illustrates the correlation of low avidity T cell and the diabetes index in mice treated with various NRP mimics. The top panel shows the percentage of low avidity NRP-A7/K^{d+} CD8⁺ cells. The middle panel shows the Kd (in nM) of each NRP mimic for the NRP-A7/K^{d+} CD8⁺ cells. The bottom panel shows the diabetes index, which takes into account both the percentage of diabetic mice in the treated group and the onset of diabetes, of mice treated with each mimic.

FIG. 28 shows (A) the percentage of CD8⁺ T cells recognizing the NRP-A7 tetramer (grey columns) or NRP-V7 tetramer (black columns) in mice treated with TUM (negative control), Ins-L, NRP-V7 or NRP-A7; and (B) the percentage of CD8⁺ T cells recognizing the Ins-L tetramer in mice treated with TUM (grey column), Ins-L (hatched column) or NRP-A7/V7 (black column).

### DETAILED DESCRIPTION OF THE INVENTION

The present invention relates to a method of selectively reducing or expanding T cells according to the antigenic specificity of the T cells. Therefore, the present invention can be used to reduce or eliminate T cells that recognize auto-antigens, such as beta cell specific T cells. As such, the present invention can be used to prevent, treat or ameliorate autoimmune diseases such as IDDM. Furthermore, the present invention can be used to expand desirable T cells, such as T cells that recognize tumor antigens, to prevent, treat and/or ameliorate diseases battled by these T cells.

Prior to describing the invention in further detail, the terms used in this application are defined as follows unless otherwise indicated.

Definitions

A “ligand”, as used herein, is a molecule capable of binding to a T cell receptor (TCR). A ligand is typically a peptide, glycopeptide, oligosaccharide, or oligonucleotide. A ligand is preferably a small organic compound with a molecular weight of 1000 or less (more preferably 500 or less).

A ligand “mimic” is an analog of a given ligand, wherein the analog is substantially similar to the ligand. “Substantially similar” means that the analog is the same as the ligand except for one or more functional groups which collectively accounts for less than about 50%, preferably less than about 40%, more preferably less than about 30%, and most preferably less than about 20%, of the molecular weight of the ligand.

A “peptide” is a organic compound comprising amino acids linked by peptide bonds. A peptide may optionally contain non-natural amino acids, modified amino acids, or moieties other than amino acids. Preferably, a peptide does not contain non-amino acid moieties.

The term “avidity” of a ligand for a T cell refers to the strength of the interaction between the ligand and the T cell, i.e., the stability of the complex formed between the ligand and the T cell. The term “affinity” refers to the stability of the complex between the ligand and a single T cell receptor (TCR). As a T cell contains a plurality of T cell receptors, the avidity of a ligand for a T cell is a function of both the affinity of the ligand for the individual TCRs and the number of TCRs on the T cell.

The “desired avidity” is an avidity selected for the intended purpose. As described herein in detail, a desired avidity for an autoimmune disease may be a low, intermediate or high avidity, depending on the dosage of the ligand displaying each avidity. The desired avidity for a tumor or pathogenic infection is typically a low avidity.

An “effective amount” is an amount sufficient to achieve the intended purpose. As described herein in detail, the effective amount, or dosage, depends on the purpose and the avidity of the ligand and can be determined according to the present disclosure.

An “auto-reactive T cell” is a T cell that recognizes an “auto-antigen”, which is a molecule produced and contained by the same individual that contains the T cell.

A “pathogenic” antigen is a molecule expressed by a pathogen, such as a bacterium, virus, fungus (including yeast), parasite, or prion.

A “tumor antigen” is a molecule expressed predominantly by a tumor cell. In particular, a tumor antigen is a molecule that can be recognized by the immune system, and such recognition can lead to death of the tumor cell expressing the tumor antigen.

A “pathogenic T cell” is a T cell that is harmful to the animal which contains the T cell.

Method

The functional consequences of T cell receptor (TCR) triggering during T cell development depend, in part, on the strength of the TCR/ligand-MHC interaction (affinity/avidity). Immature thymocytes capable of engaging ligand/MHC complexes with too high an affinity/avidity undergo death, whereas those capable of engaging ligand/MHC complexes with low affinity/avidity are signaled to differentiate into mature T-lymphocytes. Unlike immature T cells, mature T cells are thought to expand, rather than die, in response to high affinity ligands of their TCRs, unless these ligands are presented by non-professional antigen-presenting cell types (i.e., fibroblasts).

Contrary to this common belief, the present invention indicates that mature T cells are also controlled by an analogous affinity/avidity model. By screening combinatorial peptide libraries, we identified two peptide ligands for cytotoxic T cells (CTL) expressing the prevalent Vα17-MRD-Jα42 TCRα chain, which is associated with the NOD mice and IDDM. The two peptide ligands are NRP (KYNKANWFL; SEQ ID NO:1) and NRP-A7 (KYNKANAFL; SEQ ID NO:2), an alanine mutant analog of NRP with superior agonistic properties (37). These two peptides elicit the proliferation, cytokine secretion, differentiation and cytotoxicity of naive CD8⁺ T cells from 8.3-TCRαβ-transgenic NOD mice, and are recognized by a large fraction of the islet-associated CD8⁺ T cell lines (87%) and clones (45%) recruited to islets in NOD mice.

For poorly understood reasons, NOD mice start to develop insulitis at 3 wk of age, but do not begin to develop IDDM until 10 wk later. Since many of the CTL clones derived from islets of diabetic NOD mice express Vα17 and Jα42 elements joined by the N-region sequence MRD/E (see above), and these cells recognize the same peptides as the 8.3-TCR (NRP and/or NRP-A7), we hypothesized that progression of insulitis to diabetes might be driven by the accumulation of NRP/NRP-A7-reactive CTL in islets. Our studies of the fate of the NRP/NRP-A7-reactive T cell population in non-transgenic NOD mice using peptide/MHC tetramers have revealed that progression of islet inflammation to overt diabetes in the NOD mouse is driven by the “avidity maturation” of this T cell population (39). We found that, as pre-diabetic NOD mice age, their islet-associated CD8⁺ T cells contain increasing numbers of NRP-A7-reactive cells, and that these cells bind NRP-A7/K_{d} tetramers with increased avidity and longer half-lives. Importantly, repeated treatment of pre-diabetic NOD mice with soluble NRP-A7 peptide blunted the avidity maturation of the NRP-A7-reactive CD8⁺ T cell population by deleting those clonotypes expressing TCRs with the highest affinity and lowest dissociation rates for peptide/MHC binding. This inhibited the production of CTL and halted the progression from severe insulitis to diabetes. Avidity maturation of pathogenic T cell populations therefore appears to be the key event in the progression of benign inflammation to overt disease in autoimmunity.

We have found that avidity maturation of dominant T cell populations is key to the progression of T cell-mediated autoimmunity. While treatment of NOD mice with the peptide NRP-A7 blunted the avidity maturation of the NRP-reactive CD8⁺ T cell population (by eliminating the high avidity T cell subpopulation), it also promoted the expansion of the low avidity T cell subpopulation. This indicates that NRP-A7 treatment affords diabetes resistance, not simply by promoting the elimination of the high avidity T cell subpopulation, but also by promoting the expansion of the low avidity subpopulation, which has an active protective role.

Thus, the outcome of our peptide treatment experiments can be explained by an affinity/avidity model. The NRP-A7 peptide deleted the high affinity T cell subpopulation because it was able to trigger this T cell subpopulation with a strength surpassing the threshold for T cell deletion (as a result of high avidity T cells recognizing a high affinity ligand). The same peptide was able to expand the low avidity T cell subpopulation because it was able to signal the T cells' activation without reaching the threshold for deletion (as a result of low avidity T cells recognizing a high affinity ligand).

Therefore, it is possible to design peptide mimics capable of selectively triggering high and/or low avidity T cell subpopulations. Certain very high affinity mimics would delete both high and low avidity T cells, whereas very low affinity mimics would selectively expand, rather than delete, high avidity T cells and spare low avidity T cells. Peptide mimics with intermediate avidity would delete high avidity T cells while expanding low avidity T cells. The consequences of this selective triggering paradigm can be exploited for therapeutic purposes. For example, in the case of cancer, one would like to preferentially expand high avidity T cells (to enhance the anti-tumor immune response), whereas in the cause of autoimmunity it would be ideal to delete high avidity T cells and expand the protective low avidity T cells.

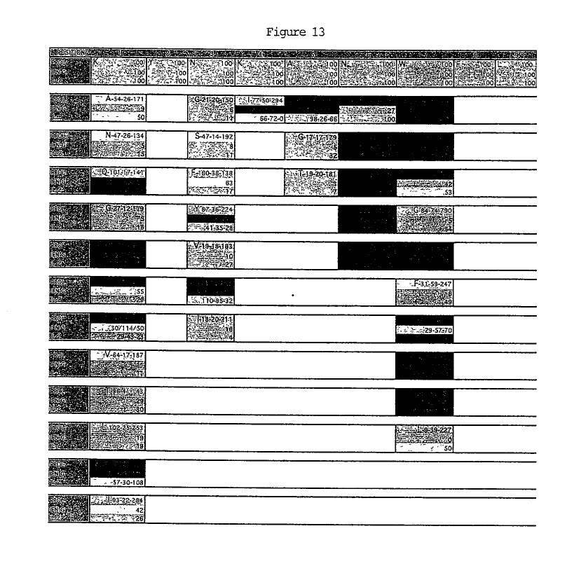

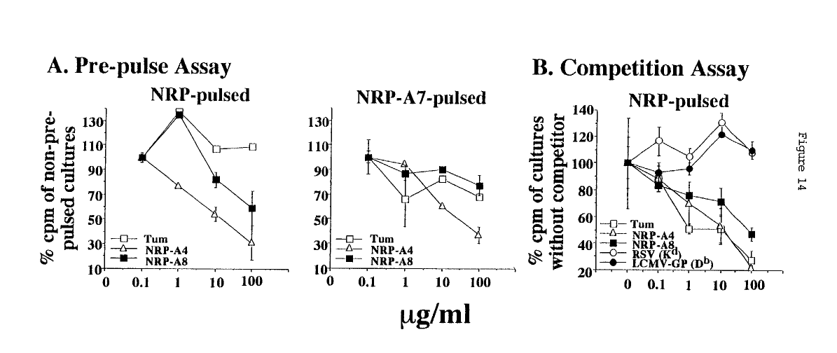

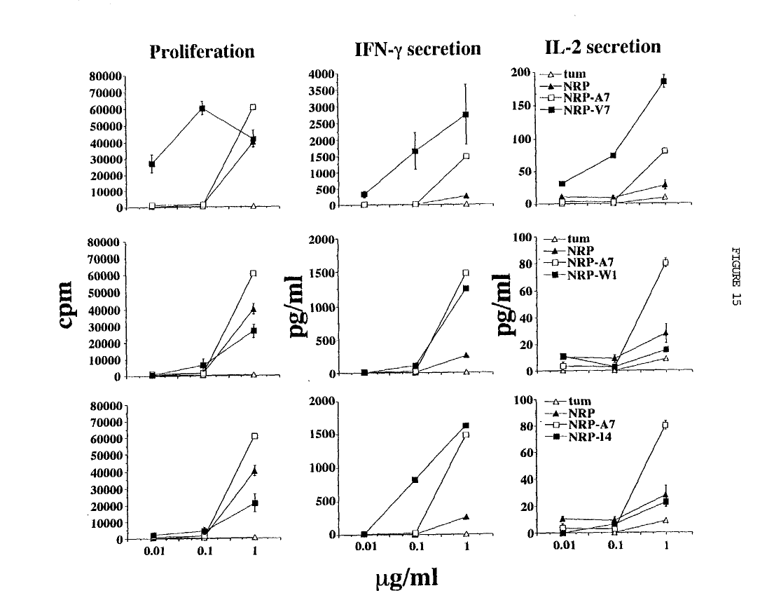

To test the validity of this hypothesis, we produced mimics of NRP capable of engaging the 8.3-TCR (a TCR that is representative of the TCRs used by NRP-reactive CD8⁺ T cells in the NOD mouse) with very low or very high affinity (when compared to NRP and NRP-A7). This was done by generating numerous single amino acid mutants of NRP (NRP mimics) and by testing the ability of each mutant to elicit proliferation, cytokine secretion and cytotoxicity of naïve or activated 8.3-CD8⁺ T cells. The results of these experiments are summarized in FIG. 13. Some of the NRP mimics that were unable to elicit the activation of naïve 8.3-CD8⁺ T cells were tested for antagonism. Antagonist mimics are unable to elicit the functional activity of T cells but antagonized the agonistic activity of functional mimics (i.e. NRP and NRP-A7). These experiments resulted in the identification of two NRP antagonists: NRP-A4 and NRP-A8 (FIG. 14). Of these, NRP-A4 was the most powerful and was chosen for further experimentation. Partial agonists and super/super-agonists were chosen among NRP mimics capable of triggering 8.3-CD8⁺ T cell activation (FIG. 13). NRP-I4 behaved as a partial agonist since it could only trigger IFN-gamma secretion by naïve 8.3-CD8⁺ T cells. NRP-V7 was chosen as a super/super-agonist since it had superior agonistic activity when compared to NRP (agonist) and NRP-A7 (super-agonist). FIG. 15 compares the agonistic properties of all these peptides on naïve 8.3-CD8⁺ T cells.

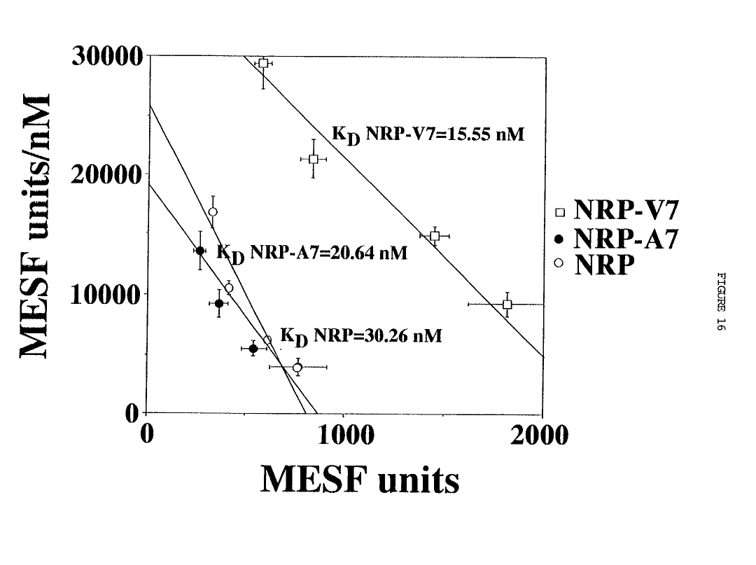

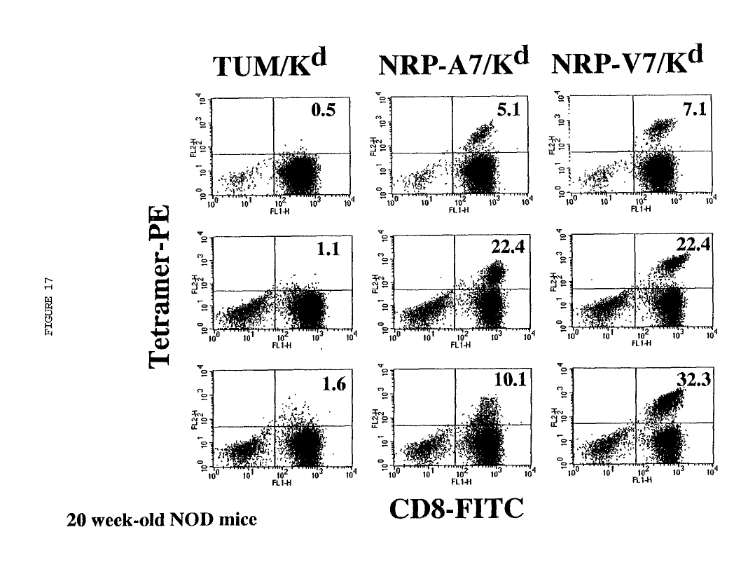

To confirm that these peptides were recognized with different avidity by 8.3-CD8⁺ T cells, we produced and tested peptide/MHC tetramers as previously described (39). A tetramer is preferably used in avidity determinations because tetramers bind more strongly than the corresponding monomers, while maintaining the same relative avidities when a plurality of ligands are compared. As shown in FIG. 16, the NRP-A4 tetramer (antagonist) interacted with the 8.3-TCR on 8.3-CD8⁺ T cells with an avidity that was too low to stably stain 8.3-CD8⁺ T cells. NRP-I4 (partial agonist), NRP (agonist), NRP-A7 (super-agonist) and NRP-V7 (super/super-agonist) bound to 8.3-CD8⁺ T cells with increased avidity (proportional to the fluorescence intensity of stained T cells). These peptides were recognized with different avidity not only by 8.3-CD8⁺ T cells, but also by the polyclonal NRP-reactive CD8⁺ T cells that can be propagated from the pancreatic islets of 20 wk-old NOD mice. As shown in FIG. 17, the NRP-V7 tetramer reacts with as many, and sometimes more islet-derived CD8⁺ T cells than the NRP-A7 tetramer, much like what we saw with the NRP and NRP-A7 tetramers (more cells bound the NRP-A7 than the NRP tetramers). As expected, the NRP-I4 tetramer bound fewer CD8⁺ T cells than the NRP tetramer. Taken together, these data demonstrated that NRP-A4, NRP-I4, NRP, NRP-A7 and NRP-V7 peptides are recognized with increasing affinity/avidity by NRP-reactive CD8⁺ T cells.

The hypothesis put forth above predicted that, in vivo, each of these peptides would trigger T cells differently depending on the T cells' avidity for the peptide (Table I):

TABLE I

| Peptide | Function | Expected Action |
|---|---|---|
| NRP-A4 | antagonist | None |
| NRP-I4 | partial agonist | Expand high affinity cells |
| NRP | moderate agonist | Moderate deletion of high affinity cells; |
| | | Moderate expansion of low affinity cells |
| NRP-A7 | superagonist | Deletion of high affinity cells; |
| | | Expansion of low affinity cells |
| NRP-V7 | super-superagonist | Deletion of high and low affinity cells |

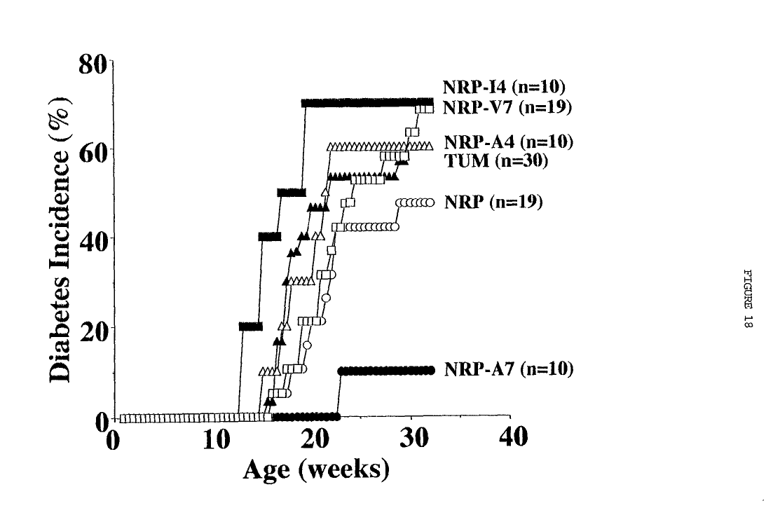

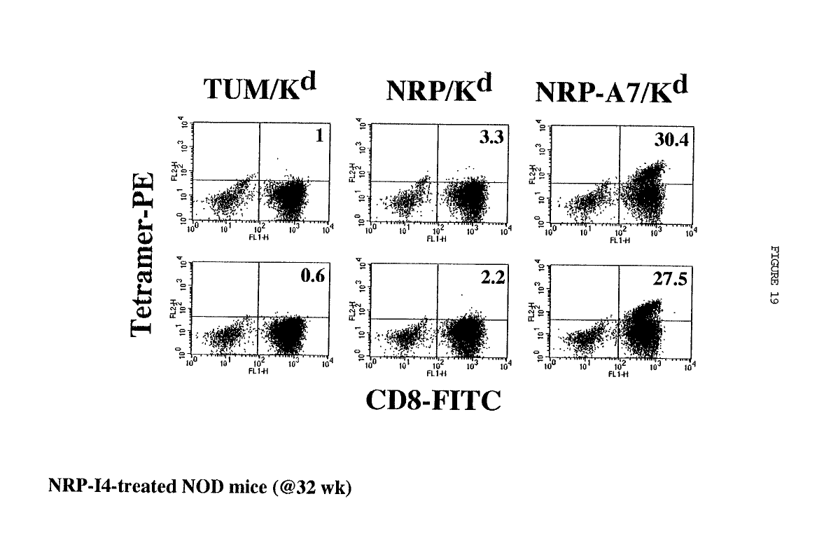

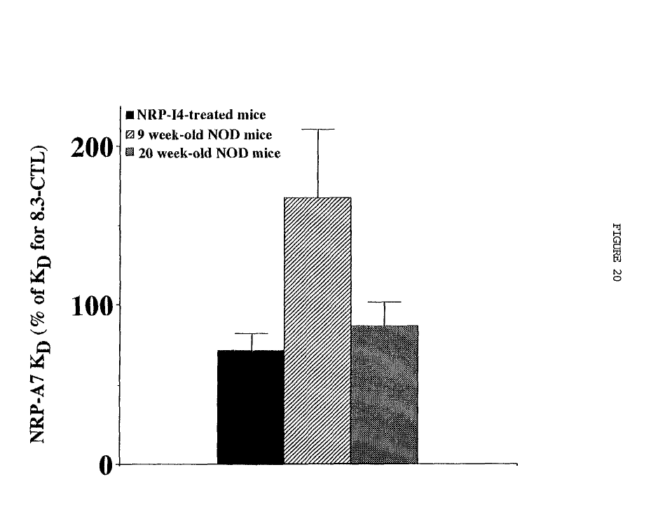

To test whether the hypothesis could in fact predict the different outcomes outlined in Table I, we treated groups of NOD female mice with repeated injections of NRP-A4, NRP-I4 and NRP-V7, and followed the mice for development of spontaneous autoimmune diabetes. T cells were also harvested from the treated mice and tested with various ligand/MHC tetramers to determine the impact of the treatment on T cell populations. As shown in FIG. 18, NRP-I4 not only did not protect the mice from diabetes, but actually caused disease acceleration. As shown in FIG. 19, the pancreatic islets of these mice contained more NRP-A7-reactive CD8⁺ T cells than untreated NOD mice, and these cells were capable of binding the NRP-A7, but not the NRP tetramer (a feature of high avidity CD8⁺ T cells, see above). Kinetic analyses in fact showed that these cells bound NRP-A7 tetramer with high avidity (FIG. 20). Therefore, as our hypothesis had predicted, NRP-I4 triggered the selective expansion of high avidity NRP-reactive CD8⁺ T cells. Since these cells have higher diabetogenic potential (see above), it is likely that their accumulation into islets is the single mechanism that accounts for the accelerated onset of diabetes seen in NRP-I4-treated mice.

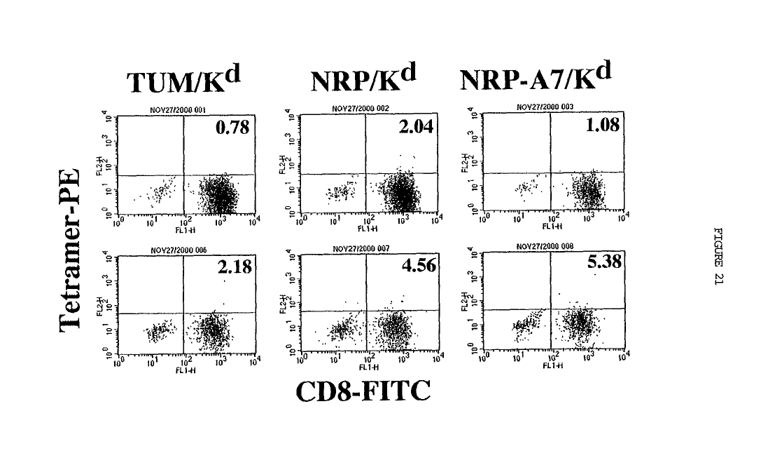

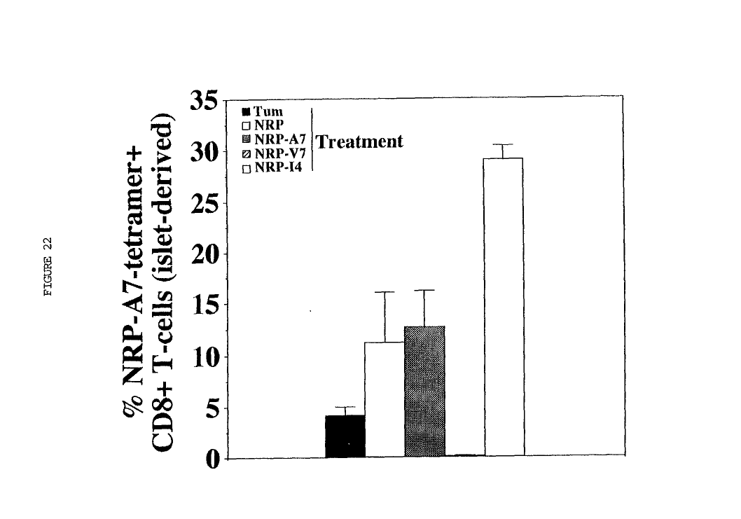

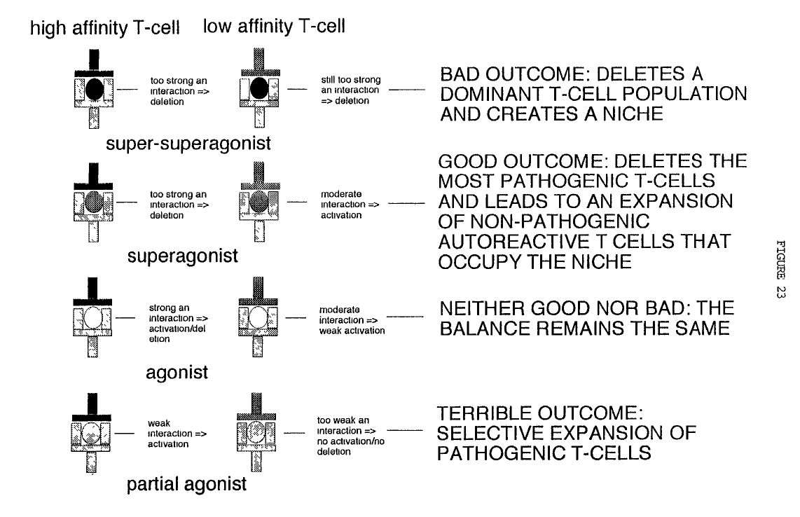

Treatment of NOD mice with the super/super-agonistic peptide NRP-V7 delayed the onset of diabetes, but did not significantly protect the mice from diabetes (FIG. 18). The islet-associated CD8⁺ T cells of NRP-V7-treated mice had virtually no NRP-reactive CD8⁺ T cells, as determined by tetramer staining (FIGS. 21 and 22). This indicates that NRP-V7 triggered the deletion of both high and low avidity NRP-reactive CD8⁺ T cells. FIG. 23 shows a summary of our interpretation of the data.

It is intriguing that NRP-V7 caused a deletion of both high and low avidity NRP-reactive CD8⁺ T cells but only delayed the onset of diabetes rather than curing the disease. We observed that NRP-V7 treated mice had a significantly increased percentage of insulin 15-23-reactive CD8⁺ T cells in the islets. Insulin 15-23-reactive cells do not recognize the NRP-based mimics, and vice versa. Without being limited to a theory, we believe that elimination of a large number of NRP-reactive CD8⁺ T cells created a “niche” that prompted the homeostatic expansion of other autoreactive T cell subsets in the islets, leading to diabetes.

Since development of spontaneous autoimmune diabetes, like many other autoimmune diseases, involves the recruitment of several different T cell specificities, these findings indicate that complete elimination of a dominant T cell population is a very inefficient way to halt the progression of an autoimmune disease. Rather, our data indicate that effective treatment of complex autoimmune diseases requires the elimination of high avidity T cell subpopulations AND the expansion of low avidity T cell subpopulations. The latter may afford protection by simply denying space to subdominant T cell populations (i.e. by occupying a niche) or by actively suppressing other pathogenic T cell populations.

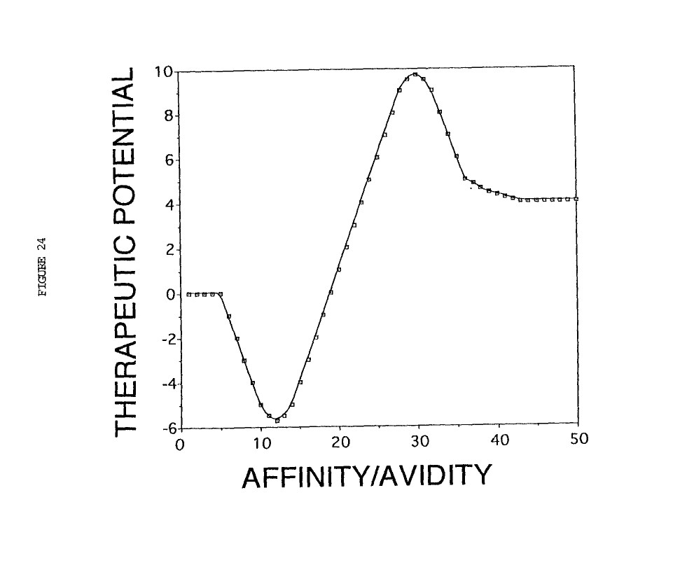

This discovery therefore provides the rationale for the design of therapeutically effective peptide mimics in diseases such as autoimmune disorders and cancer. This discovery challenges the currently prevailing model, where the affinity of a peptide for cognate TCRs is thought to be directly proportional to its potential therapeutic effect, and draws on our recent discovery that spontaneous T cell responses undergo avidity maturation. Furthermore, our model illustrates the danger of using peptides that are recognized with low avidity, for these can trigger the selective expansion of pathogenic T cells. This is currently an unrecognized phenomenon that has immediate implications for our understanding of why some currently ongoing clinical trials fail or succeed. While dangerous in the context of autoimmunity, these low affinity peptides would have therapeutic potential in the context of cancer, where activation and expansion of T cells capable of recognizing tumor antigens with high avidity would promote tumor regression. FIG. 24 illustrates a basic model for the proposed relationship between avidity/affinity of peptides for TCRs and their therapeutic potential.

In addition to avidity, the dosage of the peptide also plays a role in the effect of the peptide on clonal deletion or expansion. All the experiments described above were performed with a low dosage of peptide. We further discovered that if a high dosage of the same peptide is used, the effect shifted toward that of a higher-avidity peptide.

Thus, both low and high doses of each of TUM (negative control), NRP-A4 (low avidity), NRP-I4 (moderate avidity), NRP (intermediate avidity), NRP-A7 (high avidity) and NRP-V7 (very high avidity) were given to NOD mice and the effects were assessed (Example 11). The results (Table II) indicate that low dosage of NRP-A4 had no effect, while a high dose was anti-diabetogenic. NRP-I4 accelerated diabetes at a low dose but protected the mice from diabetes at a high dose. NRP-A7 was anti-diabetogenic at low dosage, and had no effect at high dosage. NRP-V7 was ineffective on the diabetic incidence at either low or high dosages.

TABLE II

| | | | Action |
|---|---|---|---|
| Peptide | Function/Avidity | Action (low dose) | (high dose) |
| NRP-A4 | antagonist/low | no effect | protective |
| NRP-I4 | partial agonist/ | accelerated diabetes | protective |
| | moderate | | |
| NRP | intermediate | ND | ND |
| | agonist/intermediate | | |
| NRP-A7 | superagonist/high | protective | No effect |
| NRP-V7 | super- | No effect | No effect |
| | superagonist/very | | |
| | high | | |

ND: not done

The effects of the NRP peptides on diabetes were compared to those on T cell populations, and the results indicate that peptide-induced expansion of high avidity NRP-A7-reactive CD8⁺ T-cells by low concentrations of moderate avidity peptides results in disease acceleration. They also indicate that peptide-induced expansion of low avidity NRP-A7-reactive CD8⁺ T-cells by low concentrations of high avidity peptides, or high concentrations of low avidity peptides, has an active anti-diabetogenic effect.

Thus, the present invention provides a method of preventing, treating or ameliorating a disease by selectively deleting at least one T cell that mediates the disease, or selectively expanding at least one T cell that is therapeutic for the disease. One approach to practicing the present invention is to first identify a population of T cells that recognizes the cause of the disease. These T cells are preferably the T cells that infiltrate the tissue or organ containing the auto-antigen in an autoimmune disease, the infected lesion in a pathogenic infection, or the tumor when the disease is a tumor. After the responsible T cells have been identified, a library of potential TCR ligands, particularly peptides, can be screened with the T cells in order to identify the ligand.

A series of mimics of the ligand is then prepared and the avidity of each for the T cells is determined, for example, using the tetramer analysis described herein. The avidities of all the mimics are compared, and the mimics with a desired avidity are identified. The mimics with a moderate avidity among all the mimics would be useful against an autoimmune disease, and those with a low avidity would be useful for tumors or pathogenic infections. In addition, high dosages of low avidity mimics and low dosages of high avidity mimics will also be useful in the treatment of autoimmune diseases.

Alternatively, where the antigen is known (such as the auto-antigen, tumor antigen or pathogenic antigen), the antigen can be used to identify the population of T cells involving in the pathogenesis or treatment of the disease of interest. The T cells can be identified, for example, by using a fluorescence activated cell sorter. Thereafter, mimics of the antigen can be made and screened using the T cells as described above.

It should be emphasized that in order to accurately compare the avidities of the mimics, the series of mimics should contain all the possible variants. For example, if the ligand is a peptide of 10 amino acid residues, mutational analysis should first be performed to determine which residues can be varied, using activation of the T cells of interest as an assay. Each one the variable amino acid residues thus determined is then replaced by each and every amino acid in each mimic to yield a large collection of mimics, and the avidity of every of these mimics should be determined in order to generate a complete spectrum of avidity to identify the mimics with a desired avidity.

It is contemplated that the TCR ligands useful in the present invention are not limited to peptides, as the T cell avidity maturation process applies whether the ligand is a peptide or not. For example, T cells recognizing the carbohydrate moiety of a glycoprotein auto-antigen can be the reason for an autoimmune disease. In this case, an oligosaccharide may be identified as a ligand for these pathogenic T cells, oliogosaccharide mimics can be made by methods known in the art and avidities determined according to the present invention.

Compositions

The present invention provides compositions comprising the TCR ligands identified according to the methods described herein, as well as pharmaceutical compositions comprising suitable TCR ligands useful for the prevention, treatment or amelioration of diseases or medical conditions. The pharmaceutical compositions may optionally comprise at least one pharmaceutically acceptable excipient or carrier. The pharmaceutical composition may be administered by any methods consistent with the present invention, and is preferably administered by injections. The preferred routes include intraperitoneal, intravascular, intramuscular and subcutaneous administrations.

The following examples are offered to illustrate this invention and are not to be construed in any way as limiting the scope of the present invention.

### EXAMPLES

The abbreviations used in this application have the following meanings. Abbreviations not defined have their generally accepted meanings.

| | |
|---|---|
| ° C. | = degree Celsius |
| hr | = hour |
| min | = minute |
| μM | = micromolar |
| mM | = millimolar |
| M | = molar |
| ml | = milliliter |
| μl | = microliter |
| mg | = milligram |
| μg | = microgram |
| FBS | = fetal bovine serum |
| DTT | = dithiothrietol |
| SDS | = sodium dodecyl sulfate |
| PBS | = phosphate buffered saline |
| DMEM | = Dulbecco's modified Eagle's medium |
| α-MEM | = α-modified Eagle's medium |
| β-ME | = β-mercaptoethanol |
| DMSO | = dimethylsulfoxide |
| IDDM | = insulin dependent diabetes mellitus |
| CTL | = cytotoxic T lymphocyte |
| TCR | = T cell receptor |
| RAG-2 | = recombination activating gene 2 |
| MHC | = major histocompatibility complex |
| NOD | = nonobese diabetic |
| TID | = Type-1 diabetes |

### Example 1

### K_{d}-Restricted, Beta Cell-Specific TCRβ-Transgenic NOD Mice

In initial studies, we showed that NOD islet-derived CD8⁺ T cells are beta cell-cytotoxic (29, 40), that many of these CD8⁺ CTLs are K_{d}-restricted and that they use highly homologous TCRα-CDR3 sequences (29). To investigate the role (and repertoire) of beta cell auto-antigens in the recruitment of CD8⁺ CTL in spontaneous IDDM, we produced transgenic NOD mice with the rearranged Vβ8.1⁺ TCRβ gene of a representative clone (NY8.3) (30). Transgene expression in these mice increased the peripheral frequency of beta cell-specific pre-CTL (˜10-fold) and accelerated both the recruitment of CD8⁺ CTL to islets and the onset of IDDM. Importantly, the islet-derived (but not the splenic) CD8⁺ T cells of these mice used endogenous TCRα chains identical to that of the clone donating the TCRβ transgene. These results demonstrated that CD8⁺ CTL are major effectors of beta cell damage in spontaneous IDDM and suggested that these cells accumulate in situ in response to a dominant peptide/K_{d} complex (30).

### Example 2

### K_{d}-Restricted, Beta Cell-Specific TCRαβ-Transgenic NOD Mice

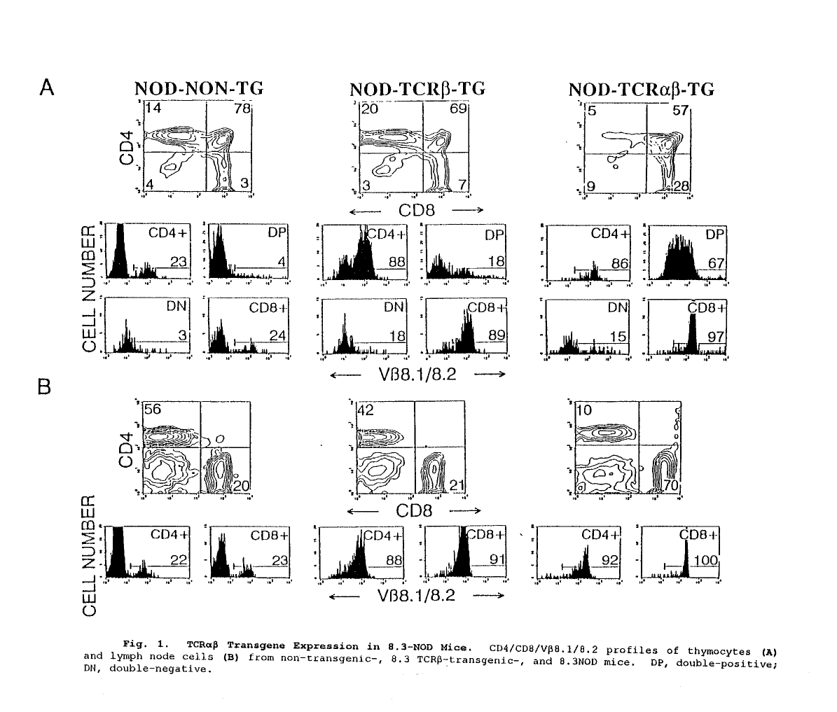

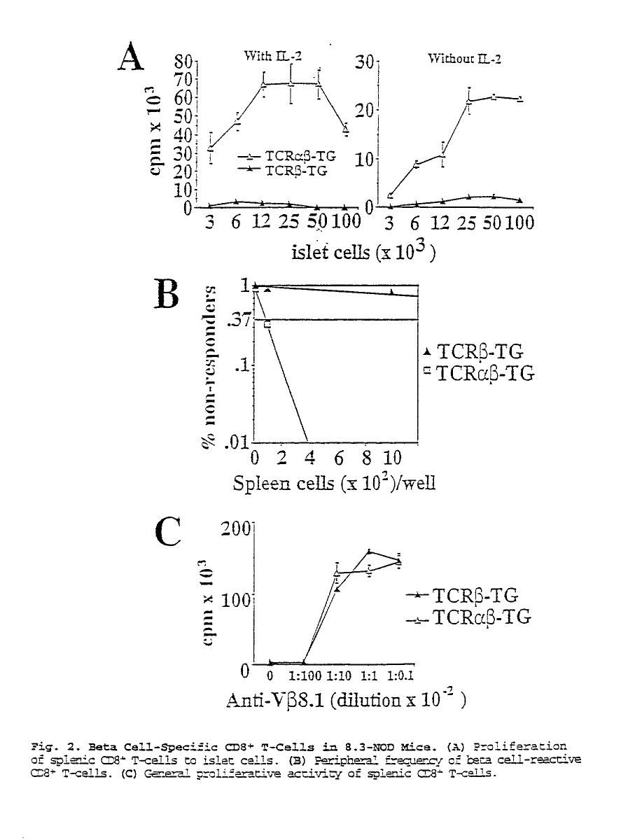

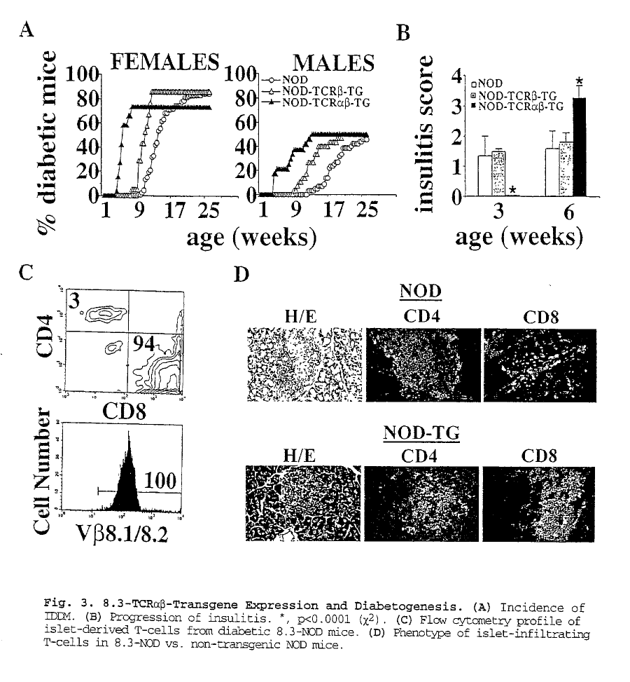

We next produced transgenic NOD mice expressing the TCRα and β chains of NY8.3 (8.3-NOD mice) (34). In 8.3-NOD, but not 8.3-TCRβ-NOD mice, thymocyte development was skewed towards the CD8⁺ subset, as expected (FIG. 1). The splenic CD8⁺ T cells of 8.3-NOD mice proliferated in response to NOD islet cells in vitro (FIG. 2A) owing to a 400-fold increase in the peripheral frequency of beta cell-reactive CD8⁺ T cells (FIG. 2B). As a result, these mice developed IDDM much earlier than NOD or 8.3-TCRβ-transgenic NOD mice (FIG. 3A), coinciding with an accelerated recruitment of CD8⁺ CTL (FIGS. 3B-D).

### Example 3

### Monoclonal T Cell NOD Mice

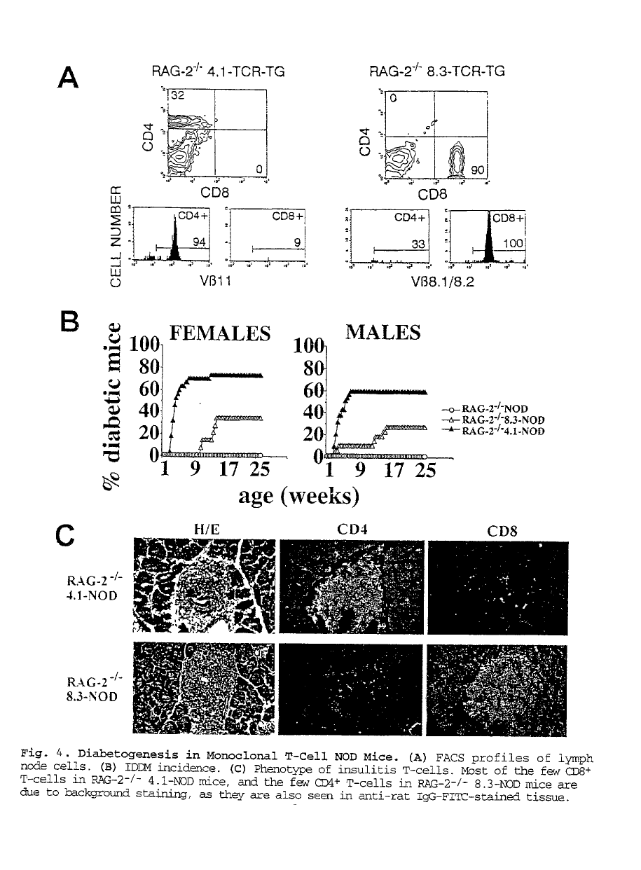

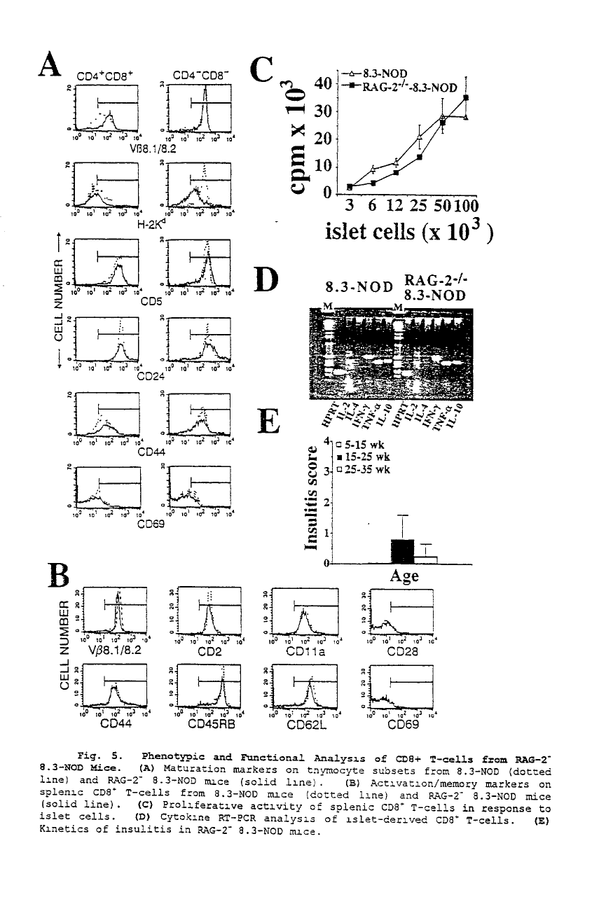

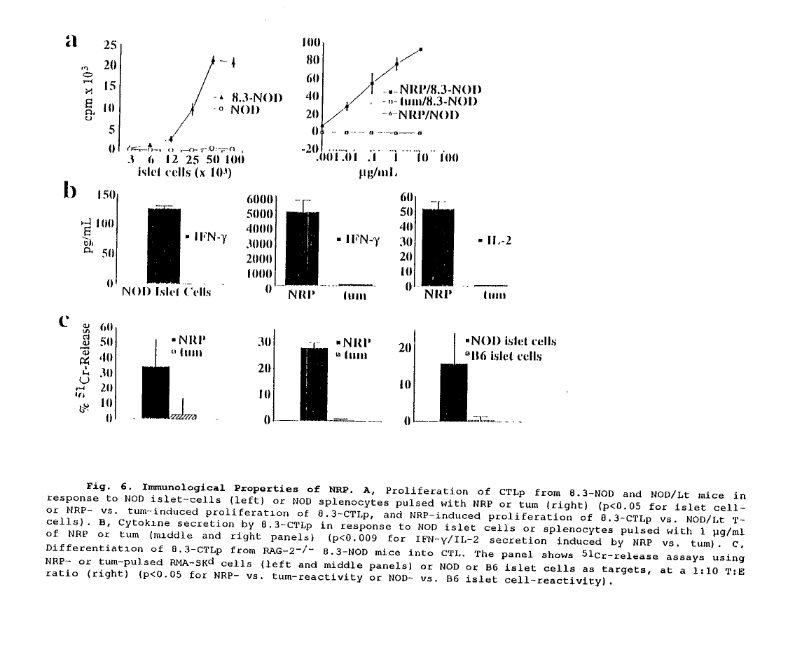

To investigate whether the CD8⁺ T cells of 8.3-NOD mice could trigger IDDM in the absence of T cells displaying endogenous TCRs, we introduced the 8.3-TCR transgenes into RAG-2^{-/-} NOD mice (34) (FIG. 4A). We also compared the ability of another highly diabetogenic, but MHC class II-restricted TCR (4.1-TCR) to trigger IDDM in the presence and absence of endogenous T cells. RAG-2^{-/-} 4.1-NOD mice developed IDDM earlier than, and as frequently as RAG-2⁺ 4.1-NOD mice. RAG-2^{-/-} 8.3-NOD mice also developed IDDM, but they did so less frequently, and later than RAG-2⁺ 8.3-NOD mice (FIG. 4B). Nearly all the islet-infiltrating T cells in the diabetic mice were either CD4⁺ (in RAG-2^{-/-} 4.1-NOD mice) or CD8⁺ (in RAG-2^{-/-} 8.3-NOD mice) (FIG. 4C). This suggested that, unlike 4.1-CD4⁺ T cells, 8.3-CD8⁺ T cells require T cell help to efficiently trigger IDDM. Additional work revealed that 8.3-CD8⁺ T cells require endogenous T cell help, not to mature properly, to proliferate in response to antigenic stimulation in vitro or to differentiate into CTLs (FIG. 5), but rather to accumulate efficiently into islets (Table III). These findings have led us to propose that, in non-transgenic NOD mice, recruitment of 8.3-like autoreactive CD8⁺ T cells into islets is preceded and/or accompanied by the recruitment of certain highly pathogenic CD4⁺ T cells.

TABLE III
Non-Transgenic CD4⁺ T cells Can Trigger the Recruitment and Activation
of Autoreactive CD8⁺ T cells in RAG-2^{-/-} 8.3-NOD Mice

| | | | | B220⁺ | CD4⁺ | CD8⁺ |
|---|---|---|---|---|---|---|
| | Recipient | IDDM | Insulitis | Cells in | Cells in | Cells in |
| Cells | (n) | (n) | (n) ** | Lesion | Lesion | Lesion |
| CD8⁺- | RAG-2^{-/-} | 0/7 | 0 (3) | ND | ND | ND |
| depleted | NOD (7) | | | | | |
| * | | | | | | |
| | RAG-2^{-/-} | 4/5 | 3.2 ± 0.3 (4) | +++ | ++ | +++ |
| | 8.3-NOD | | | | | |
| | (5) | | | | | |
| CD4⁺ | | 2/2 | 3.4 ± 0.2 (2) | +/− | +++ | +++ |
| *** | | | | | | |

* Splenocytes from 4-5 week old non-transgenic female NOD mice were depleted of CD8⁺ T cells using anti-CD8 Mab (53-6.7)-coated magnetic beads, as described (26). 8 × 10⁶ cells (containing <0.5% CD8⁺ T cells) were injected into 5-8 week-old female mice.
** 8-10 weeks after transfer.
*** Splenic T cells from non-transgenic female NOD mice were enriched for CD4⁺ T cells (>92%) and depleted of CD8⁺ T cells and B220⁺, P4/80⁺, Mac-1⁺ and 33D4⁺ cells (<.04%) using mAb-coated immunobeads and affinity columns. Each mouse received 15 × 10⁶ cells.
ND, not determined.

### Example 4

### Prevalent CD8⁺ T Cell Response to One Peptide/K_{d} Complex in IDDM (NRP)

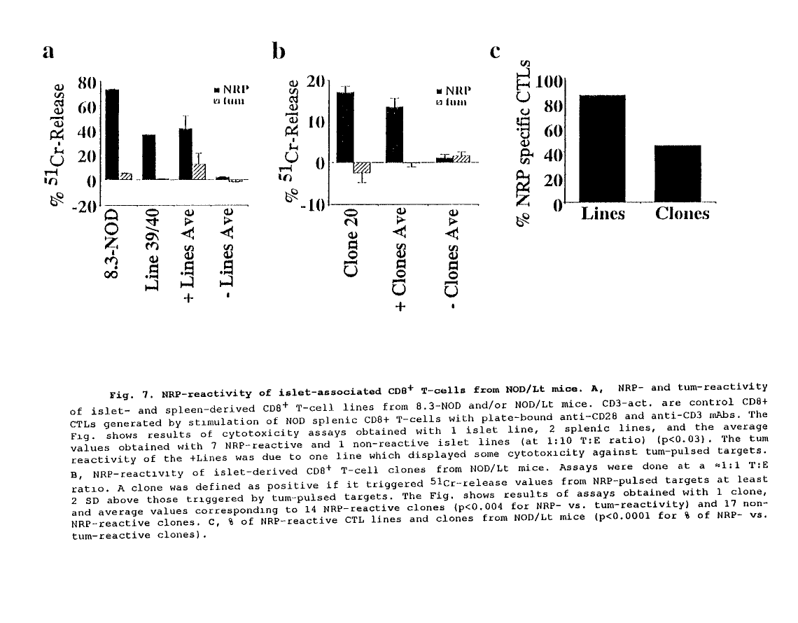

To test the hypothesis that the CD8⁺ T cell response in murine IDDM is driven by a prevalent peptide/K_{d} complex, we set out to determine the molecular nature of peptide(s) targeted by 8.3-CD8⁺ CTLs, which express the prevalent Vα17-MRD/E-Jα42 chain. Studies employing combinatorial peptide libraries as a source of antigen and K_{d} cDNA-transfected RMA-S cells (RMA-S-K_{d}) as targets resulted in the identification of a target peptide (KYNKANWFL) (NRP). As shown in FIG. 6, NRP had the same agonistic properties as the natural beta cell auto-antigen on naive 8.3-CD8⁺ T cells.

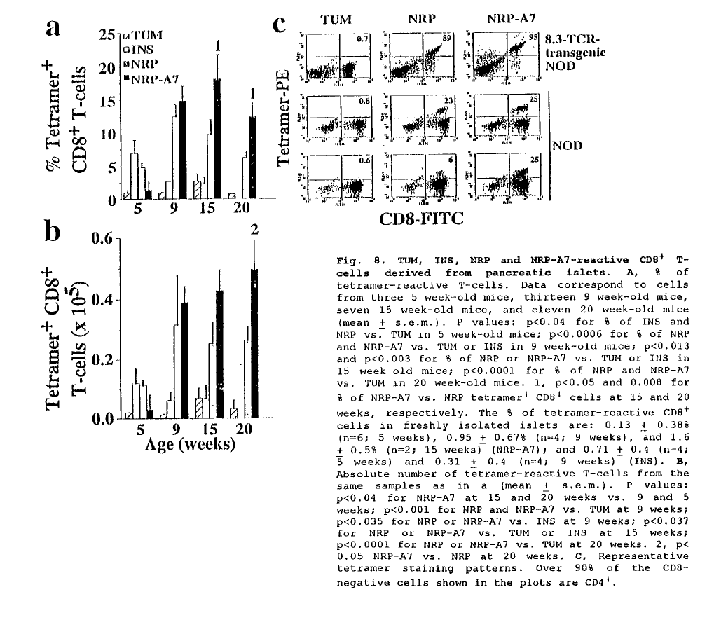

To see whether the beta cell-specific CD8⁺ T cell response in NOD mice is predominantly directed against this peptide/K_{d} complex, as our previous studies had suggested, we asked whether CD8⁺ T cells propagated from islets of acutely diabetic NOD mice could recognize NRP. As shown in FIG. 7A, most of these lines (7 of 8—87.5%—, FIG. 7C) killed NRP-pulsed RMA-SK_{d} cells in vitro. In contrast, TUM, the negative control peptide, pulsed cells were not killed. To confirm that the islet associated CD8⁺ T cells of most NOD mice contain clones capable of recognizing the NRP/K_{d} complex, we generated 31 CD8⁺ CTL clones from islets of 9 NOD mice, and tested the clones' ability to kill NRP- or TUM-pulsed RMA-SK_{d} cells. Of the 31 clones tested, 14 (45%) were cytotoxic against NRP-, but not TUM-pulsed, RMA-SK_{d} targets (FIGS. 7B and C). These data showed that CD8⁺ T cells mount a prevalent response against one peptide/MHC class I complex in NOD mice, and suggested that NRP-reactive CD8⁺ T cells might play a critical role in diabetogenesis.

### Example 5

### Detection of NRP-Reactive CD8⁺ T Cells in NOD Mice Using Peptide/MHC Tetramers

In order to follow the accumulation of antigen-specific CD8⁺ T cells in islets of pre-diabetic NOD mice, we generated H-2K_{d} tetramers containing the peptides NRP, NRP-A7, TUM or INS [LYLVCGERG; an insulin-derived peptide recognized by islet-associated T cells from young NOD mice (38)]. The NRP and NRP-A7 tetramers stained virtually all the splenic CD8⁺ T cells from 8.3-TCRαβ-transgenic NOD mice, but <1% of the splenic CD8⁺ T cells from NOD mice. The INS tetramer stained the INS-reactive CTL clone G9C8 (38), but neither this tetramer nor the negative control tetramer (TUM) stained the splenic CD8⁺ T cells from NOD or 8.3-TCRαβ-transgenic NOD mice (data not shown).

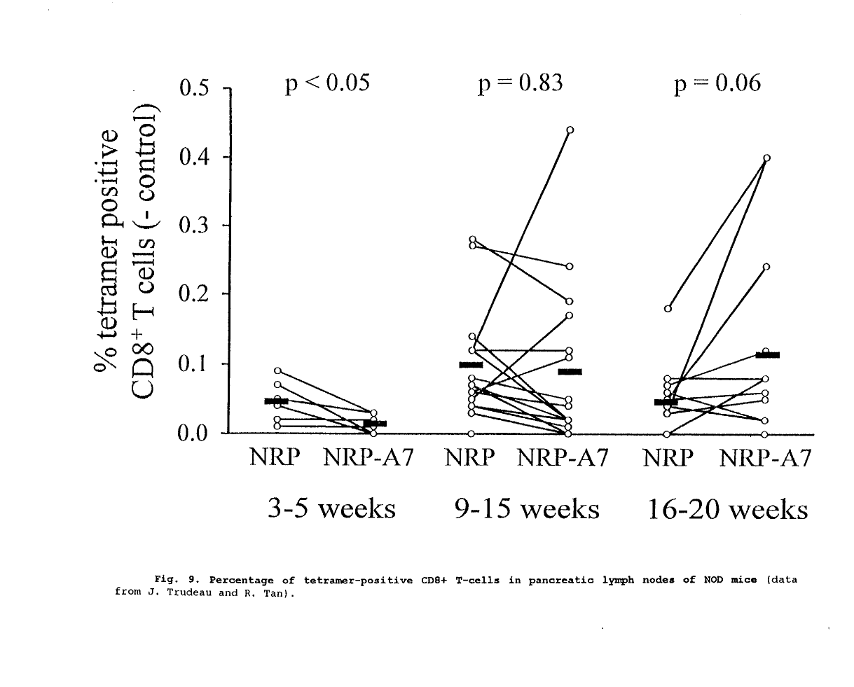

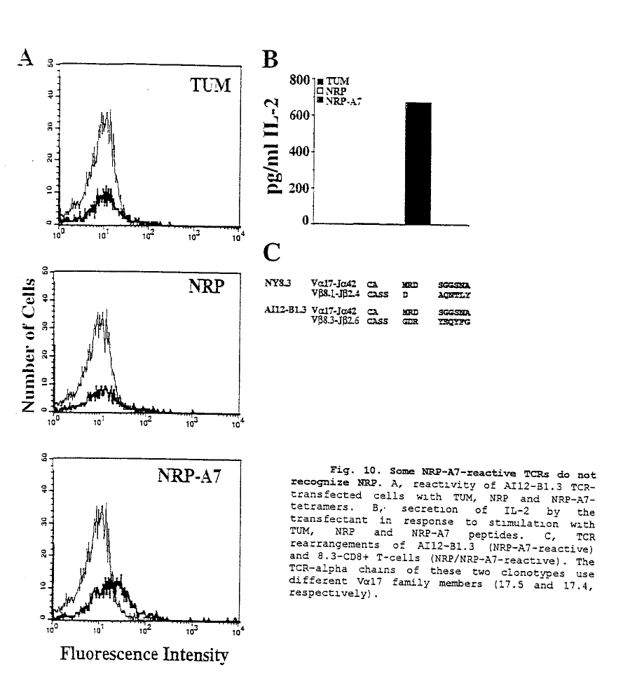

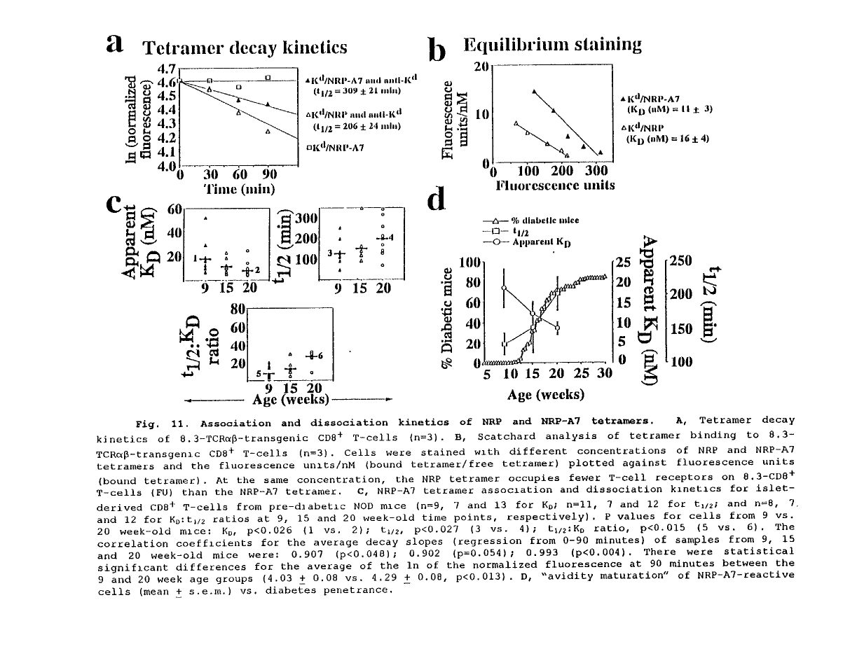

Since tetramer-reactive cells represent a very small fraction of all the islet-associated T cells, we analyzed the presence of tetramer-reactive clonotypes within the cell population that has undergone antigen-driven activation and thus is likely to contain autoreactive specificities. Islets from non-diabetic 5, 9, 15 and 20 wk-old NOD mice were cultured in the presence of rIL-2 for 6-7 d, to selectively expand IL-2R-positive T cells [˜10% of all the islet-associated CD8⁺ T cells (30)]. There were age-dependent increases in the number of T cells recovered from NOD islets and in the % of CD8⁺ T cells contained within lines. Tetramer staining indicated that NRP/NRP-A7-reactive CD8⁺ T cells constitute a significant fraction of the in vivo-activated CD8⁺ T cells contained within islets (FIGS. 8A, B). Notably, the size of the NRP-A7-reactive CD8⁺ T cell population increased with age, reaching ˜18% by 15 wk (FIGS. 8A, B). This increase was antigen-specific, since the number of NRP/NRP-A7-reactive CD8⁺ T cells was significantly greater than the number of INS (and TUM)-reactive CD8⁺ T cells at all ages tested, except 5 weeks (FIGS. 8A, B). Surprisingly, the number of NRP-A7-reactive CD8⁺ T cells at 20 wk was significantly greater than the number of NRP-reactive CD8⁺ T cells (FIGS. 8A, B). This was not an artifact of in vitro culture since pancreatic lymph node cells from 20 wk-old, but not 3-5 wk-old NOD mice bind the NRP-A7 tetramer more efficiently than the NRP tetramer (FIG. 9). Since most, if not all the NRP-reactive lines are also NRP-A7-reactive, but not vice versa (FIG. 8C), and since at least one of the Vα17-MRD-Jα42-expressing T cell clones isolated by DiLorenzo et al. (36) recognizes NRP-A7 but not NRP (FIG. 10), the above data suggested that the age-dependent increase in the size of the NRP/NRP-A7-reactive population is accompanied by an outgrowth of clones capable of recognizing NRP-A7 but not NRP. Furthermore, since NRP/NRP-A7-reactive clonotypes (i.e. 8.3-CTL) bind the NRP-A7 tetramer with longer half-life and higher avidity (lower K_{D}) than the NRP tetramer (FIGS. 11A and 11B), we reasoned that the outgrowing clonotypes might be those with the highest avidity for NRP-A7/H-2K_{d} binding.

### Example 6

### Avidity Maturation of the NRP-Reactive CD8⁺ T Cell Population in IDDM

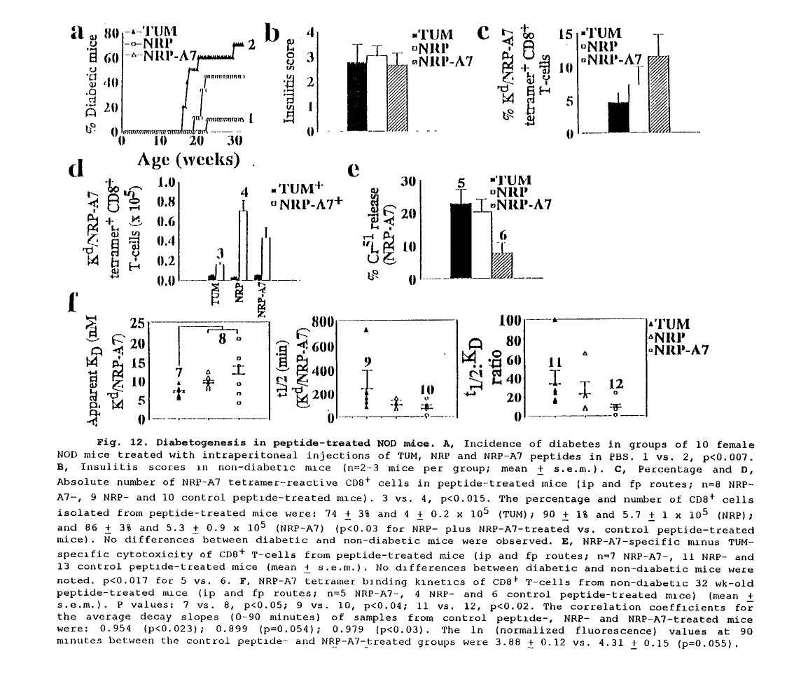

To test this hypothesis, we measured the association (K_{D}) and dissociation kinetics ™1/2) of the interaction between NRP-A7 tetramers and islet-derived CD8⁺ T cells obtained at different ages (9, 15 and 20 wk). As shown in FIG. 11C, CD8⁺ T cells isolated from 20 wk-old NOD mice bound the NRP-A7 tetramer with higher avidity and longer half-life than CD8⁺ T cells isolated from 9 wk-old mice. The islet-associated NRP-A7-reactive CD8⁺ T cell population therefore increases its avidity for peptide/MHC as the mice approach the age at which the incidence of IDDM follows an exponential distribution (FIG. 11D). To determine whether development of IDDM in NOD mice requires the accumulation of NRP-A7-reactive CTL in islets, cohorts of female NOD mice were treated with repeated injections of 100 μg of TUM, NRP or NRP-A7 in PBS i.p. Previous studies in 8.3-TCRαβ-transgenic NOD mice had indicated that repeated administration of NRP or NRP-A7 via the i.p. and f.p. routes induced the deletion of 8.3-CD8⁺ T cells (unpublished data). As shown in FIG. 12A, TUM-treated NOD mice developed IDDM with an incidence similar to untreated NOD mice (FIG. 11D). NOD mice treated with NRP developed IDDM at a slightly lower rate when compared to TUM-treated mice (FIG. 12A). In contrast, mice treated with NRP-A7 were highly protected (FIG. 12A). Therefore, NRP-A7-reactive CD8⁺ T cells play a critical role in IDDM.

To ascertain whether the NRP-A7 treatment had induced the deletion of NRP-A7-reactive CD8⁺ T cells, we examined islets of peptide-treated, non-diabetic 32 wk-old mice for the presence of this T cell population. Pathological studies revealed that TUM-, NRP- and NRP-A7-treated mice displayed similar insulitis scores (FIG. 12B), and that their insulitis lesions contained similar % of CD4⁺ and CD8⁺ T cells (not shown). Surprisingly, the islet-derived CD8⁺ T cells of NRP-A7- and NRP-treated mice contained greater numbers of NRP-A7 tetramer-reactive cells than control peptide-treated mice (FIGS. 12C and 11D), refuting the notion that the NRP-A7-reactive T cell population had undergone massive deletion in NRP-A7-treated mice. However, the CD8⁺ T cells derived from NRP-A7-treated mice were less cytotoxic to NRP-A7-pulsed, RMA-SK_{d} targets than those derived from NRP- or control peptide-treated mice (FIG. 12E), indicating that NRP-A7 treatment had somehow impaired the cytotoxic potential of the NRP-A7-reactive population. To determine whether this resulted from the absence of clonotypes bearing high affinity TCRs for NRP-A7, we compared the kinetics of NRP-A7 tetramer binding to CD8⁺ T cells derived from peptide-treated mice. As shown in FIG. 12F, CD8⁺ T cells derived from NRP-A7-treated mice bound the NRP-A7 tetramer with a lower avidity and shorter half-life than CD8⁺ T cells derived from NRP- or control peptide-treated mice. Hence, treatment with NRP-A7 had selectively eliminated those NRP-A7-reactive CD8⁺ T cells expressing high affinity TCRs for NRP-A7, while allowing the local expansion of clonotypes bearing lower affinity TCRs. Since treatment with NRP-A7 also halted the progression from benign to pernicious islet inflammation, we concluded that development of IDDM in NOD mice requires the avidity maturation of the NRP-A7-reactive CD8⁺ T cell population.

### Example 7

### Functional Testing of NRP Mimics

To test the validity of this hypothesis, we produced mimics of NRP capable of engaging the 8.3-TCR (a TCR that is representative of the TCRs used by NRP-reactive CD8⁺ T cells in the NOD mouse) with very low or very high affinity (when compared to NRP and NRP-A7). This was done by generating numerous single amino acid mutants of NRP (NRP mimics) and by testing the ability of each mutant to elicit proliferation, cytokine secretion and cytotoxicity of naïve or activated 8.3-CD8⁺ T cells. The results of these experiments are summarized in FIG. 13. Some of the NRP mimics that were unable to elicit the activation of naïve 8.3-CD8⁺ T cells were tested for antagonism. Antagonist mimics are unable to elicit the functional activity of T cells but antagonized the agonistic activity of functional mimics (i.e. NRP and NRP-A7). The figure shows the amino acids that are tolerated at each position, as well as the functional response elicited by the corresponding NRP variants (shown as the percentage of the response obtained with NRP, at the top of the figure). The percentage values correspond to the responses induced by each peptide at 1, 0.1 and 0.01 μM or at 1 μM (for peptides which did not induce measurable responses at 0.1 and 0.01 μM). Peptide-induced responses are color-coded from low, medium, high, high and very high (blue, yellow, green and red, respectively). The data are representative of 3 different experiments.

These experiments resulted in the identification of two NRP antagonists: NRP-A4 and NRP-A8 (FIG. 14). Of these, NRP-A4 was the most powerful and was chosen for further experimentation. Partial agonists and super/super-agonists were chosen among NRP mimics capable of triggering 8.3-CD8⁺ T cell activation (FIG. 13). NRP-I4 behaved as a partial agonist since it could only trigger IFN-gamma secretion by naïve 8.3-CD8⁺ T cells. NRP-V7 was chosen as a super/super-agonist since it had superior agonistic activity when compared to NRP (agonist) and NRP-A7 (super-agonist). FIG. 15 compares the agonistic properties of all these peptides on naïve 8.3-CD8⁺ T cells.

### Example 8

### Functional Testing of Peptide/MHC Tetramers

To confirm that these peptides were recognized with different avidity by 8.3-CD8⁺ T cells, we produced and tested peptide/MHC tetramers. As shown in FIG. 16, the NRP-A4 tetramer (antagonist) interacted with the 8.3-TCR on 8.3-CD8⁺ T cells with an avidity that was too low to stably stain 8.3-CD8⁺ T cells. NRP-I4 (partial agonist), NRP (agonist), NRP-A7 (super-agonist) and NRP-V7 (super/super-agonist) bound to 8.3-CD8⁺ T cells with increased avidity (proportional to the fluorescence intensity of stained T cells). These peptides were recognized with different avidity not only by 8.3-CD8⁺ T cells, but also by the polyclonal NRP-reactive CD8⁺ T cells that can be propagated from the pancreatic islets of 20 wk-old NOD mice. As shown in FIG. 17, the NRP-V7 tetramer reacts with as many, and sometimes more islet-derived CD8⁺ T cells than the NRP-A7 tetramer, much like what we saw with the NRP and NRP-A7 tetramers (more cells bound the NRP-A7 than the NRP tetramers). As expected, the NRP-I4 tetramer bound fewer CD8⁺ T cells than the NRP tetramer. Taken together, these data demonstrated that NRP-A4, NRP-I4, NRP, NRP-A7 and NRP-V7 peptides are recognized with increasing affinity/avidity by NRP-reactive CD8⁺ T cells.

### Example 9

### In Vivo Testing of NRP and NRP Peptide Mimics

The hypothesis put forth above predicted that, in vivo, each of these peptides would trigger T cells differently depending on the T cells' avidity for NRP. This is summarized in Table I. To test whether the hypothesis could in fact predict the different outcomes outlined in Table I, we treated groups of NOD female mice with repeated injections of NRP-A4, NRP-I4 and NRP-V7, and followed the mice for development of spontaneous autoimmune diabetes. As shown in FIG. 18, NRP-I4 not only did not protect the mice from diabetes, but actually caused disease acceleration. As shown in FIG. 19, the pancreatic islets of these mice contained more NRP-A7-reactive CD8⁺ T cells than untreated NOD mice, and these cells were capable of binding the NRP-A7, but not the NRP tetramer (a feature of high avidity CD8⁺ T cells, see above). Kinetic analyses in fact showed that these cells bound NRP-A7 tetramer with high avidity (FIG. 20). Therefore, as predicted, NRP-I4 triggered the selective expansion of high avidity NRP-reactive CD8⁺ T cells. Since these cells have higher diabetogenic potential (see above), it is likely that their accumulation into islets is the single mechanism that accounts for the accelerated onset of diabetes seen in NRP-I4-treated mice.

### Example 10

### In Vivo Testing of the Super/Super Agonist Peptide NRP-V7

Treatment of NOD mice with the super/super-agonistic peptide NRP-V7 delayed the onset of diabetes, but did not significantly protect the mice from diabetes (FIG. 18). The islet-associated CD8⁺ T cells of NRP-V7-treated mice had virtually no NRP-reactive CD8⁺ T cells, as determined by tetramer staining (FIGS. 21 and 22). This indicated that NRP-V7 triggered the deletion of both high and low avidity NRP-reactive CD8⁺ T cells. FIG. 23 shows a summary of our interpretation of the data.

Since development of spontaneous autoimmune diabetes, like many other autoimmune diseases, involves the recruitment of several different T cell specificities, these findings indicate that complete elimination of a dominant T cell population is a very inefficient way to halt the progression of an autoimmune disease. Rather, our data implies that effective treatment of complex autoimmune diseases requires the elimination of high avidity T cell subpopulations AND the expansion of low avidity T cell subpopulations. The latter may afford protection by simply denying space to subdominant T cell populations (i.e. by occupying a niche) or by actively suppressing other pathogenic T cell populations.

### Example 11

### Comparison of Low and High Doses of Peptide Ligands

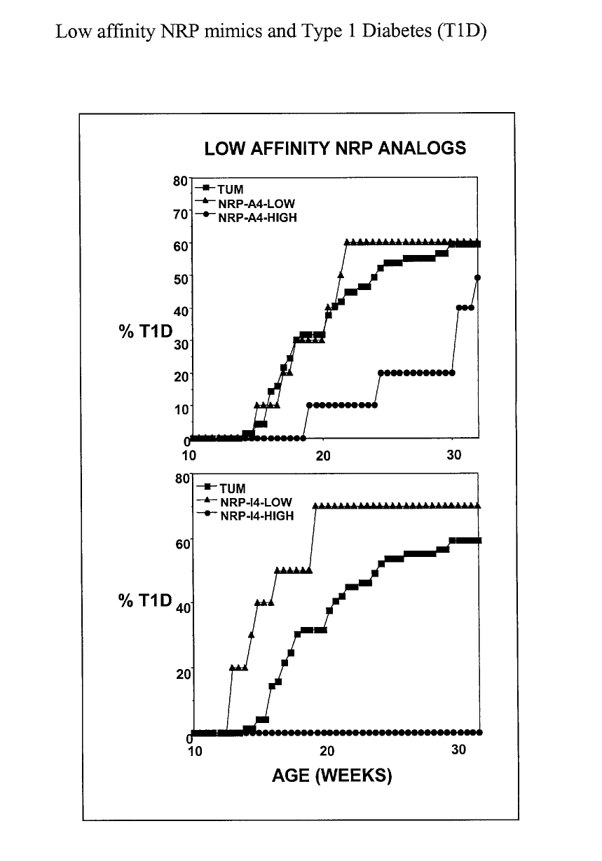

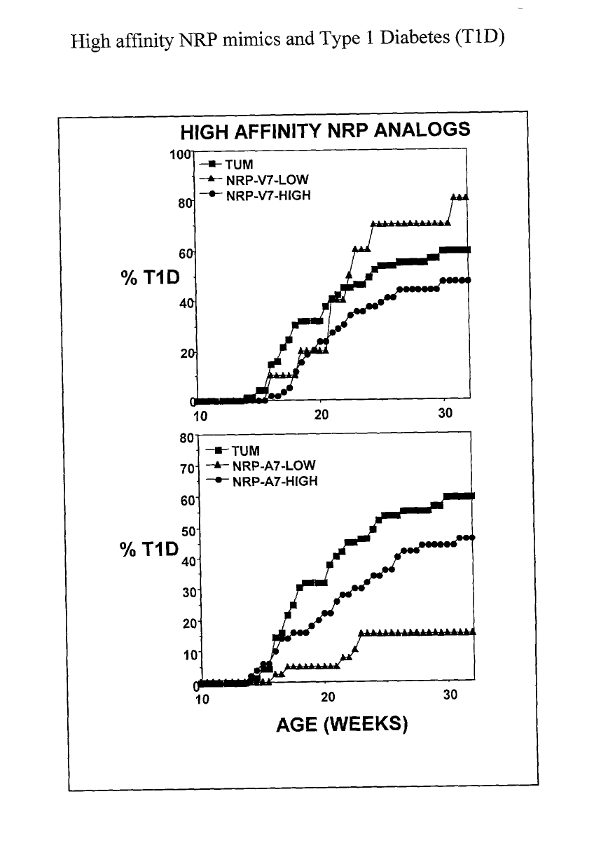

To test the effect of dosage of the peptide ligands on T cell clonal delection or expansion, we treated groups of NOD female mice with repeated injections of “low” or “high” doses of TUM (negative control), NRP, NRP-A7, NRP-A4, NRP-I4 and NRP-V7. Low concentrations of peptide were prepared by resuspending 100 mg of dry peptide in PBS in the absence of acids. The peptides in these preparations, which were also used in previous Examples, are aggregated, and the effective concentrations were low. Soluble, high concentrations of peptide were prepared by diluting 100 mg of soluble peptide (solubilized in the presence of acid) in PBS. The mice were followed for development of spontaneous autoimmune diabetes.

Whereas NRP-A4 had no anti- or pro-diabetogenic effect when given at a low concentration, it had a significant protective effect when given at a higher concentration (FIG. 25). In contrast, NRP-I4 not only did not protect the mice from diabetes, but actually accelerated the disease process, when given at a low concentration (FIG. 26). When administered at a high concentration, however, NRP-I4 was completely protective, as none of 10 treated mice developed diabetes (FIG. 25). The high avidity peptide NRP-A7 had a significant protective effect when given at a low concentration, but was rather ineffective when given at a higher concentration (FIG. 26).

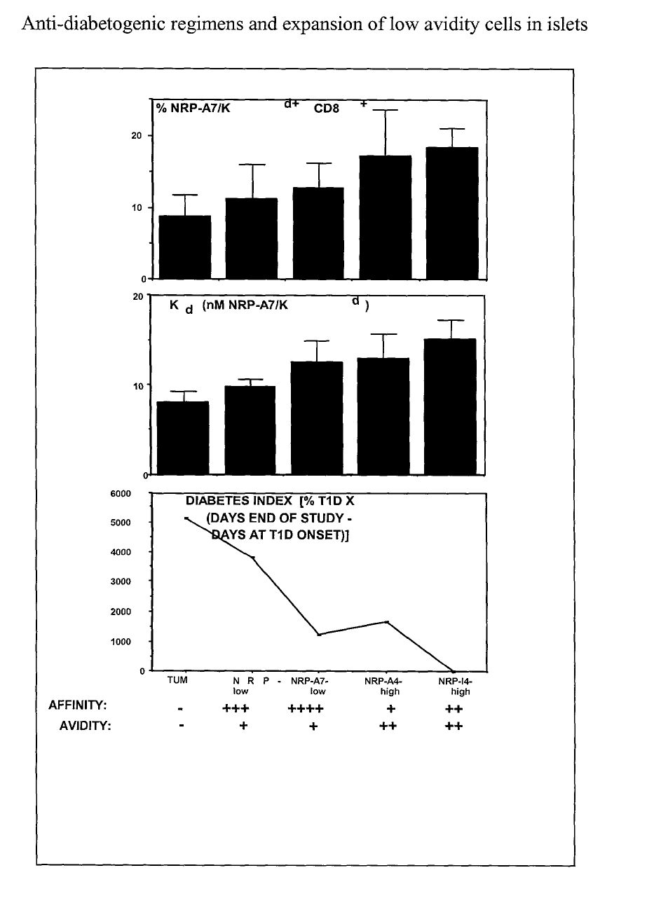

The effect of the peptides on T cell populations was also determined. Comparison of the tetramer-binding kinetics of the islet-associated CD8⁺ T-cells of mice which had received anti-diabetogenic regimens (high concentrations of NRP-A4 and NRP-I4, and low concentrations of NRP-A7) indicated that protection (as measured by calculating the “diabetes index”, which is a function of both, the age at onset and the incidence of diabetes) was correlated with presence of increasing percentages of low avidity (high K_{D}) NRP-A7-reactive CD8⁺ T-cells in islets (FIG. 27). Interestingly, the mice which had received low doses of NRP-I4 also contained significantly increased numbers of NRP-A7 tetramer-reactive CD8⁺ T-cells in islets (>25%). However, unlike the NRP-A7-reactive CD8⁺ T-cells that accumulate in islets of NRP-A7-treated mice (39), the islet-associated NRP-A7-reactive cells of NRP-I4-treated mice bound tetramer with high avidity (data not shown).

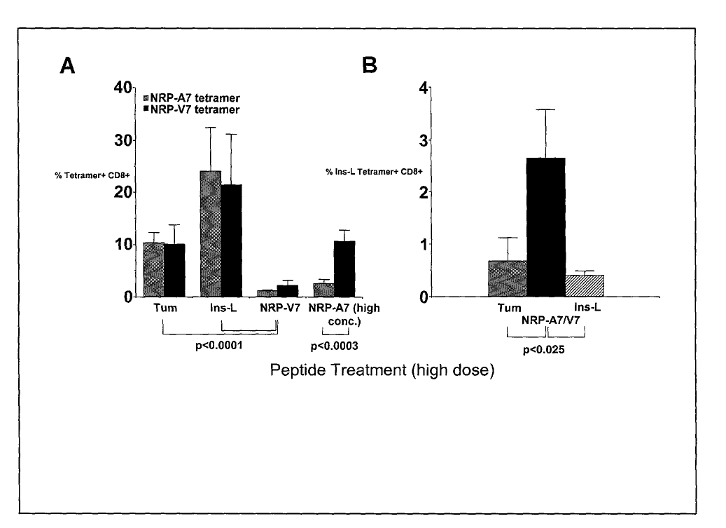

Taken together, these data indicate that peptide-induced expansion of high avidity NRP-A7-reactive CD8⁺ T-cells by low concentrations of moderate avidity peptides results in disease acceleration. They also indicate that peptide-induced expansion of low avidity NRP-A7-reactive CD8⁺ T-cells by low concentrations of high avidity peptides or high concentrations of low avidity peptides has an active anti-diabetogenic effect, possibly by promoting the occupation of intra-islet “space” by cells that cannot effectively damage beta cells (owing the low affinity of their TCRs for peptide/MHC).

Whereas low concentrations of NRP-A7 fostered the accumulation of low avidity NRP-A7-reactive T-cells in islets, leading to diabetes resistance, high concentrations of NRP-A7, and both low and high concentrations of NRP-V7, caused a nearly complete deletion of all NRP-A7-reactive cells (FIG. 28), but did not efficiently protect the mice from diabetes (FIG. 26). Surprisingly, the islet-associated CD8⁺ T-cells of these mice also contained significantly increased percentages of insulin 15-23 tetramer-reactive CD8⁺ T-cells. Since insulin 15-23-reactive cells do not recognize NRP-based mimics and vice versa, these results suggest that elimination of the NRP-A7-reactive population created a “niche” that prompted the homeostatic expansion of other autoreactive T-cell subsets in islets, leading to diabetes.

The invention is not limited to the embodiments set forth in the above examples.

We claim:

1. A method for selectively expanding or deleting at least one T cell from a T cell population, comprising:
(a) providing a ligand that binds to at least one T cell in said T cell population with a desired avidity; and
(b) contacting said T cell population with an effective amount of said ligand under conditions wherein the T cells that bind to said ligand with an avidity higher than the desired avidity are deleted, the T cells that bind to said ligand with an avidity lower than the desired avidity are expanded, and the T cells that do not bind said ligand are unaffected.

2. The method of claim 1 wherein said ligand is prepared by the steps comprising:
(i) providing a test ligand which is recognized by said at least one T cell in said T cell population;
(ii) preparing a series of ligand mimics based on said test ligand;
(iii) determining the binding avidity of said test ligand and said series of ligand mimics to said at least one T cell; and
(iv) selecting the ligand mimics in said series of ligand mimics that bind to said at least one T cell with the desired avidity.

3. The method of claim 1 wherein the T cells deleted in step (b) are auto-reactive T cells.

4. The method of claim 3 wherein the auto-reactive T cells mediate insulin-dependent diabetes mellitus (IDDM).

5. The method of claim 4 wherein the ligand is selected from the group consisting of NRP-A4, NRP-I4, NRP, NRP-A7 and NRP-V7.

6. The method of claim 1 wherein the T cells expanded in step (b) recognize pathogenic or tumor antigens.

7. The method of claim 1 wherein the ligand is a peptide.

8. A method of preventing, ameliorating or treating an autoimmune disease which is caused by at least one auto-reactive T cell in a mammal, comprising:
(a) providing a ligand which binds to said at least one auto-reactive T cell with a desired avidity; and
(b) administering to said mammal an effective amount of said ligand under conditions wherein the T cells which bind to said ligand with an avidity higher than the desired avidity are deleted, the T cells which bind to said ligand with an avidity lower than the desired avidity are expanded, and the T cells which do not bind said ligand are unaffected.

9. The method of claim 8 wherein said ligand is prepared by steps comprising:
(i) providing a test ligand which is recognized by said at least one auto-reactive T cell;
(ii) preparing a series of ligand mimics based on said test ligand;
(iii) determining the binding avidity of said test ligand and said series of ligand mimics to said at least one auto-reactive T cell; and
(iv) selecting the ligand mimics in said series of ligand mimics which bind to said at least one auto-reactive T cell with the desired avidity.

10. The method of claim 8 wherein said autoimmune disease is IDDM.

11. The method of claim 10 wherein the ligand is selected from the group consisting of NRP-A4, NRP-I4, NRP, NRP-A7 and NRP-V7.

12. The method of claim 8 wherein the ligand is a peptide.

13. A composition useful for selectively expanding a T cell clone or deleting a T cell clone, comprising a ligand which binds to at least one T cell in a T cell population with a desired avidity;
wherein contacting said T cell population with an effective amount of said ligand results in deletion of the T cells which bind to said ligand with an avidity higher than the desired avidity, and expansion of the T cells which bind to said ligand with an avidity lower than the desired avidity, while T cells which do not bind said ligand are unaffected.

14. The composition of claim 13 wherein the ligand is a peptide.

15. The composition of claim 14 wherein the peptide is selected from the group consisting of NRP-A4, NRP-I4, NRP, NRP-A7 and NRP-V7.

16. A pharmaceutical composition for preventing, ameliorating or treating a disease by selectively expanding or deleting a T cell, comprising a pharmaceutically acceptable excipient and an effective amount of a ligand which binds to at least one T cell in a T cell population with a desired avidity;
wherein contacting said T cell population with an effective amount of said ligand results in deletion of the T cells which bind to said ligand with an avidity higher than the desired avidity, and expansion of the T cells which bind to said ligand with an avidity lower than the desired avidity, while T cells which do not bind said ligand are unaffected.

17. The pharmaceutical composition of claim 16 wherein the ligand is a peptide.

18. The pharmaceutical composition of claim 17 wherein the peptide is selected from the group consisting of NRP-A4, NRP-I4, NRP-A7 and NRP-V7.
